(19) United States
(12) Patent Application Publication
(10) Pub. No.: US 2012/0151636 A1
(43) Pub. Date: Jun. 14, 2012

(54) Peptides for Stimulating Plant Disease Resistance

(75) Inventors: Pamela Ronald, Davis, CA (US); Sang-Wook Han, Davis, CA (US); Sang-Won Lee, Woodland, CA (US)

(73) Assignee: The Regents of the University of California, Oakland (US)

(21) Appl. No.: 13/263,733

(22) PCT Filed: Apr. 6, 2010
(86) PCT No.: PCT/US10/30036

§ 371 Date: Feb. 24, 2012

Related U.S. Application Data
(62) Division of application No. 61/167,621, filed on Apr. 8, 2009.

(51) Int. Cl. A01H 5/00; A01H 5/10; C12N 1/19; C12N 1/15; C12N 1/21; C07K 7/08; C12N 5/10

(52) U.S. Cl. 800/320.2; 530/326; 800/298; 435/419; 435/254.11; 435/252.3; 435/254.2; 435/348; 435/325

(57) ABSTRACT

Peptides that stimulate plant disease resistance are described.

### CROSS-REFERENCE TO RELATED PATENT APPLICATIONS

The present patent applications claims benefit of priority to U.S. Provisional Patent Application No. 61/167,621, filed on Apr. 8, 2009, which is incorporated by reference for all purposes.

### STATEMENT AS TO RIGHTS TO INVENTIONS MADE UNDER FEDERALLY SPONSORED RESEARCH AND DEVELOPMENT

The US Government has a paid-up license in this invention and the right in limited circumstances to require the patent owner to license others on reasonable terms as provided for by the terms of Grant No. GM059962, awarded by the National Institutes of Health.

### BACKGROUND OF THE INVENTION

Innate immunity provides a first line of defense against pathogen attack and is activated rapidly following infection. In contrast to the adaptive immune system that depends on somatic gene rearrangements for the generation of antigen receptors with random specificities, the innate immune system uses a set of defined receptors for pathogen recognition (Girardin, S. E. et al., Trends Microbiol. 10:193 (2002)). While it is now widely appreciated that pathogen recognition receptors (PRRs) play a key role in innate immunity in plants and animals, very little is known about the pathogen-associated molecular patterns (PAMPs), also called MAMPs (microbe-associated molecular patterns) recognized by such receptors.

In animals, recognition of PAMPs at the cell surface is largely carried out by the Toll-like receptor (TLR) family that contains leucine rich repeats (LRRs) in the extracellular domain and a Toll-interleukin receptor intracellular domain (Werling, D. et al., Vet. Immunol. Immunopathol. 91:1 (2003)). Although TLRs recognize diverse molecules, they activate a common signaling pathway via association with non-RD (arginine-aspartic acid) kinases to induce a core set of defense responses (Barton, G. M. et al., Science 300:1524 (2003)). In plants, cell surface recognition of PAMPs is carried out by receptor-like kinases that also fall into the non-RD class (ca. 47 in Arabidopsis and 371 in rice) (Dardick, C. et al., PLoS Pathog. 2:e2 (2006)).

Representative PAMPs recognized by plant and animal cell surface PRRs include flagellin, a proteinaceous component of bacterial polar flagella [recognized by human TLR5 and Arabidopsis flagellin-sensitive 2 (FLS2); (Gomez-Gomez, L. et al., Trends Plant Sci. 7:251 (2002); Hayashi, F. et. al., Nature 410:1099 (2001))], lipopolysaccharide of Gram-negative bacteria [recognized by TLR4; (Hoshino, K. et al., J. Immunol. 162:3749 (1999))], the elongation factor-Tu [recognized by elongation factor Tu receptor (EFR), (Kunze, G. et al., Plant Cell 16:3496 (2004))], and a peptidoglycan of Gram-positive bacteria (Leulier, F. et al., Nat. Immunol. 4:478 (2003)). For some PAMPs, post-translational modifications such as glycosylation (Pseudomonas aeruginosa) or acylation (Yersina pestis) can affect the specificity of PAMP-PRR recognition (Che, F. S. et al., J. Biol. Chem. 275:32347 (2000); Lapaque, N. et al., Cell Microbiol. 8:401 (2006); Tunkel, C. et al., Mol. Microbiol. 58:289 (2005)).

Given the abundance of animal TLRs and the non-RD class of plant cell surface receptor kinases and their predicted importance in innate immunity and host defense, there is great interest in identifying the PAMPs that they detect and the post-translational modifications controlling their host specificity.

The rice PRR, XA21, confers resistance to strains of the Gram-negative bacteria Xanthomonas oryzae pv. oryzae (Xoo) that express a predicted PAMP, designated AvrXA21. Xa21 codes for a predicted cell-surface localized receptor-like kinase consisting of an extracellular LRR domain, a transmembrane domain, and a non-RD cytoplasmic kinase domain (Tunkel, C. et al., Mol. Microbiol. 58:289 (2005); Song, W. et al., Science 270:1804 (1995)). Because identification of the PAMP that XA21 recognizes could have significant impact toward understanding this large but poorly understood class of receptors, we have directed a major effort towards isolation of this molecule.

Previous studies, using genetic approaches, led to the identification of six Xoo genes, falling into two functional classes, which are required for AvrXA21 (rax) activity. The first class consists of 3 genes (raxA, raxB and raxC) that encode components of a bacterial type I secretion system (TOSS). The complex generated by these three proteins is thought to form a pore through which molecules are actively transported (Thanabalu, T. et al., Embo J. 17:6487 (1998)).

The second class of rax genes includes raxP and raxQ, which encode an adenosine-5′-triphosphate sulfurylase and adenosine-5′-phosphosulfate kinase. These proteins function in concert to produce 3′-phosphoadenosine 5′-phosphosulfate (PAPS) (Shen, Y. et al., Mol. Microbiol. 44, 37 (2002)), the universal sulfuryl group donor. This class also includes RaxST, which encodes a protein showing similarity with mammalian and bacterial sulfotransferases. RaxST is predicted to catalyze transfer of the sulfuryl-group from PAPS to a specific substrate. Xoo strains carrying mutations in any of these 6 rax genes no longer trigger XA21-mediated resistance.

### Definitions

“Enhanced disease resistance” refers to an increase in the ability of a plant to prevent pathogen infection or pathogen-induced symptoms. Enhanced resistance can be increased resistance relative to a particular pathogen species or genus or can be increased resistance to all pathogens (e.g., systemic acquired resistance).

The term “promoter” refers to regions or sequence located upstream and/or downstream from the start of transcription and which are involved in recognition and binding of RNA polymerase and other proteins to initiate transcription. A “plant promoter” is a promoter capable of initiating transcription in plant cells. A plant promoter can be, but does not have to be, a nucleic acid sequence originally isolated from a plant.

The term “plant” includes whole plants, shoot vegetative organs/structures (e.g. leaves, stems and tubers), roots, flowers and floral organs/structures (e.g. bracts, sepals, petals, stamens, carpels, anthers and ovules), seed (including embryo, endosperm, and seed coat) and fruit (the mature ovary), plant tissue (e.g. vascular tissue, ground tissue, and the like) and cells (e.g. guard cells, egg cells, trichomes and the like), and progeny of same. The class of plants that can be used in the method of the invention is generally as broad as the class of higher and lower plants amenable to transformation techniques, including angiosperms (monocotyledonous and dicotyledonous plants), gymnosperms, ferns, and multicellular algae. It includes plants of a variety of ploidy levels, including aneuploid, polyploid, diploid, haploid and hemizygous.

A polynucleotide sequence is “heterologous to” an organism or a second polynucleotide sequence if it originates from a foreign species, or, if from the same species, is modified from its original form. For example, a promoter operably linked to a heterologous coding sequence refers to a coding sequence from a species different from that from which the promoter was derived, or, if from the same species, a coding sequence which is not naturally associated with the promoter (e.g. a genetically engineered coding sequence or an allele from a different ecotype or variety).

“Recombinant” refers to a human manipulated polynucleotide or a copy or complement of a human manipulated polynucleotide. For instance, a recombinant expression cassette comprising a promoter operably linked to a second polynucleotide may include a promoter that is heterologous to the second polynucleotide as the result of human manipulation (e.g., by methods described in Sambrook et al., Molecular Cloning—A Laboratory Manual, Cold Spring Harbor Laboratory, Cold Spring Harbor, N.Y., (1989) or Current Protocols in Molecular Biology Volumes 1-3, John Wiley & Sons, Inc. (1994-1998)). In another example, a recombinant expression cassette may comprise polynucleotides combined in such a way that the polynucleotides are extremely unlikely to be found in nature. For instance, human manipulated restriction sites or plasmid vector sequences may flank or separate the promoter from the second polynucleotide. One of skill will recognize that polynucleotides can be manipulated in many ways and are not limited to the examples above.

“Pathogens” include, but are not limited to, viruses, bacteria, nematodes, fungi or insects (see, e.g., Agrios, Plant Pathology (Academic Press, San Diego, Calif. (1988)).

The term “nucleic acid” or “polynucleotide” as used herein refers to a deoxyribonucleotide or ribonucleotide in either single- or double-stranded form. The term encompasses nucleic acids containing known analogues of natural nucleotides which have similar or improved binding properties, for the purposes desired, as the reference nucleic acid. The term also includes nucleic acids which are metabolized in a manner similar to naturally occurring nucleotides or at rates that are improved for the purposes desired. The term also encompasses nucleic-acid-like structures with synthetic backbones. DNA backbone analogues provided by the invention include phosphodiester, phosphorothioate, phosphorodithioate, methylphosphonate, phosphoramidate, alkyl phosphotriester, sulfamate, 3′-thioacetal, methylene(methylimino), 3′-N-carbamate, morpholino carbamate, and peptide nucleic acids (PNAs); see Oligonucleotides and Analogues, a Practical Approach, edited by F. Eckstein, IRL Press at Oxford University Press (1991); Antisense Strategies, Annals of the New York Academy of Sciences, Volume 600, Eds. Baserga and Denhardt (NYAS 1992); Milligan (1993) J. Med. Chem. 36:1923-1937; Antisense Research and Applications (1993, CRC Press). PNAs contain non-ionic backbones, such as N-(2-aminoethyl)glycine units. Phosphorothioate linkages are described in WO 97/03211; WO 96/39154; Mata (1997) Toxicol. Appl. Pharmacol. 144:189-197. Other synthetic backbones encompassed by the term include methyl-phosphonate linkages or alternating methylphosphonate and phosphodiester linkages (Strauss-Soukup (1997) Biochemistry 36: 8692-8698), and benzylphosphonate linkages (Samstag (1996) Antisense Nucleic Acid Drug Dev 6: 153-156).

The phrase “host cell” refers to a cell from any organism. Preferred host cells are derived from plants, bacteria, yeast, fungi, insects or other animals. Methods for introducing polynucleotide sequences into various types of host cells are well known in the art.

An “expression cassette” refers to a nucleic acid construct, which when introduced into a host cell (e.g., a plant cell), results in transcription and/or translation of a RNA or polypeptide, respectively.

Two nucleic acid sequences or polypeptides are said to be “identical” if the sequence of nucleotides or amino acid residues, respectively, in the two sequences is the same when aligned for maximum correspondence as described below. The term “complementary to” is used herein to mean that the sequence is complementary to all or a portion of a reference polynucleotide sequence.

One example of algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

“Percentage of sequence identity” is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “substantial identity” of polynucleotide sequences means that a polynucleotide comprises a sequence that has at least 25% sequence identity. Alternatively, percent identity can be any integer from 25% to 100%, e.g., at least: 25%, 30%, 35%, 40%, 45%, 50%, 55%, 60%, 65%, 70%, 75%, 80%, 85%, 90%, 95%, or 99% compared to a reference sequence using the programs described herein; preferably BLAST using standard parameters, as described below. One of skill will recognize that the percent identity values above can be appropriately adjusted to determine corresponding identity of proteins encoded by two nucleotide sequences by taking into account codon degeneracy, amino acid similarity, reading frame positioning and the like. Substantial identity of amino acid sequences for these purposes normally means sequence identity of at least 40%., e.g., any integer from 40% to 100%. Exemplary embodiments include at least 40%, 45%, 50%, 55%, 60%, 65%, 70%, 75%, 80%, 85%, 90%, 95%, or 99%. In some embodiments, polypeptides that are “substantially similar” share sequences as noted above except that residue positions which are not identical may differ by conservative amino acid changes. Accordingly, the present invention provides polynucleotides encoding a polypeptide comprising an amino acid sequence substantially identical across the whole length of SEQ ID NO:1 or SEQ ID NO:2. The present invention also provides for a polypeptide comprising an amino acid sequence substantially identical across the whole length of SEQ ID NO:1 or SEQ ID NO:2. Conservative amino acid substitutions refer to the interchangeability of residues having similar side chains.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)
(see, e.g., Creighton, Proteins (1984)).

As defined herein, the term “exogenous application” taken in its broadest context includes contacting or administering cells, tissues, organs or organisms with a suitable compound or element. The compound may be applied to a plant in a suitable form for uptake (such as through application to the soil for uptake via the roots, or by applying directly to the leaves, for example by spraying).

As defined herein, the term “sulfated tyrosine” is used to include tyrosine-O-sulfate residues comprising a sulfate group covalently bound via the hydroxyl group of the tyrosine side chain. Alternatively, tyrosine may be O-sulfated at a terminal carboxyl group. Sulfate may be added to a tyrosine by post-translational modification of a peptide or protein by incorporation of an optionally protected sulfotyrosine building block during peptide synthesis, by chemical synthesis, or by chemical alteration, for example. As used herein, “Y” indicates a tyrosine residue, while “Y*” indicates a sulfated tyrosine.

### BRIEF SUMMARY OF THE INVENTION

The present invention provides an isolated or purified polypeptide comprising A(E/Q)(N/G)LSY*N(F/Y)(V/A)(E/G)(G/A/S)DY(V/A)(R/K)T(P/D/K) (SEQ ID NO:2), wherein Y* represents a sulfated tyrosine and wherein the amino acids in parentheses are options at the designated position. In some embodiments, the polypeptide consists of A(E/Q)(N/G)LSY*N(F/Y)(V/A)(E/G)(G/A/S)DY(V/A)(R/K)T(P/D/K) (SEQ ID NO:2), wherein Y* represents a sulfated tyrosine. In some embodiments, the polypeptide comprises AENLSY*NFVEGDYVRTP (SEQ ID NO:1), wherein Y* represents a sulfated tyrosine.

In some embodiments, the polypeptide consists of AENLSY*NFVEGDYVRTP (SEQ ID NO:1), wherein Y* represents a sulfated tyrosine.

In some embodiments, the polypeptide, when contacted to a rice plant expressing XA21, enhances disease resistance in the plant compared to a control plant not contacted with the polypeptide.

The present invention further provides for compositions comprising the isolated or purified polypeptides as described above or otherwise provided herein. In some embodiments, the composition is an agricultural formulation. In some embodiments, the agricultural formulation further comprises an agriculturally suitable carrier, surfactant, herbicide, fungicide, pesticide, or fertilizer.

The present invention also provides for methods of making the polypeptide as described above or otherwise herein. In some embodiments, the method comprising purifying a polypeptide from a mixture comprising a cell that comprises an expression cassette, wherein the expression cassette comprises a promoter operably linked to a polynucleotide, the polynucleotide encoding the polypeptide, wherein the polypeptide comprises A(E/Q)(N/G)LSY*N(F/Y)(V/A)(E/G)(G/A/S)DY(V/A)(R/K)T(P/D/K) (SEQ ID NO:2), wherein Y* is optionally sulfated.

In some embodiments, the polypeptide comprises AENLSY*NFVEGDYVRTP (SEQ ID NO:1), wherein Y* represents a sulfated tyrosine.

In some embodiments, the cell is a bacterial, fungal, yeast, plant, insect or animal cell.

In some embodiments, the purified polypeptide comprises a sulfated Y* and the cell further comprises one or more enzyme that sulfates the Y tyrosine in the polypeptide. In some embodiments, the purified polypeptide comprises an unsulfated Y and the method further comprises sulfating the Y tyrosine in the polypeptide following the purifying step. In some embodiments, the method comprises contacting the polypeptide with one or more enzyme that sulfates the Y tyrosine in the polypeptide.

The present invention also provides methods of enhancing disease resistance in a plant. In some embodiments, the method comprises contacting the plant with a sufficient amount of the polypeptide as described above or elsewhere herein such that disease resistance of the plant is enhanced compared to disease resistance of a control plant that is not contacted by the polypeptide.

In some embodiments, the polypeptide comprises AENLSY*NFVEGDYVRTP (SEQ ID NO:1), wherein Y* represents a sulfated tyrosine.

In some embodiments, the plant expresses XA21. In some embodiments, the plant is a rice plant.

The present invention also provides plants contacted with an exogenous application of the polypeptide as described above or elsewhere herein. In some embodiments, the plant is a seed. In some embodiments, the plant is a rice plant. In some embodiments, the plant expresses XA21.

The present invention provides a plant comprising a heterologous expression cassette, the expression cassette comprising a promoter operably linked to a polynucleotide, the polynucleotide encoding a polypeptide comprising A(E/Q)(N/G)LSYN(F/Y)(V/A)(E/G)(G/A/S)DY(V/A)(R/K)T(P/D/K) (SEQ ID NO:3).

In some embodiments, the polypeptide consists of A(E/Q)(N/G)LSYN(F/Y)(V/A)(E/G)(G/A/S)DY(V/A)(R/K)T(P/D/K) (SEQ ID NO:3). In some embodiments, the polypeptide comprises AENLSYNFVEGDYVRTP (SEQ ID NO:4). In some embodiments, the polypeptide consists of AENLSYNFVEGDYVRTP (SEQ ID NO:4).

In some embodiments, the plant expresses the polypeptide and the polypeptide comprises a sulfated tyrosine. In some embodiments, the plant has enhanced disease resistance in the plant compared to a control plant not comprising the expression cassette. In some embodiments, the plant expresses an XA21 polypeptide. In some embodiments, the XA21 polypeptide is heterologous to the plant.

The present invention also provides isolated host cells comprising a heterologous expression cassette, the expression cassette comprising a promoter operably linked to a polynucleotide, the polynucleotide encoding a polypeptide comprising A(E/Q)(N/G)LSYN(F/Y)(V/A)(E/G)(G/A/S)DY(V/A)(R/K)T(P/D/K) (SEQ ID NO:3). In some embodiments, the polypeptide consists of A(E/Q)(N/G)LSYN(F/Y)(V/A)(E/G)(G/A/S)DY(V/A)(R/K)T(P/D/K) (SEQ ID NO:3). In some embodiments, the polypeptide comprises AENLSYNFVEGDYVRTP (SEQ ID NO:4). In some embodiments, the polypeptide consists of AENLSYNFVEGDYVRTP (SEQ ID NO:4).

In some embodiments, the host cell expresses the polypeptide and the polypeptide comprises a sulfated tyrosine. In some embodiments, the cell is selected from the group consisting of a plant cell, a fungal cell, a bacterial cell, a yeast cell, a insect cell and a mammalian cell.

### BRIEF DESCRIPTION OF THE DRAWINGS

FIG. 1. Isolation of Ax21 (A) Reverse phase-high pressure liquid chromotagraphy elution profile of peptides secreted from Xoo strain PXO99 (carrying Ax21 activity). Peptide-enriched samples from the PXO99 supernatant were separated on a reverse phase C18 column (1×250 mm, flow rate: 0.05 mL/min) with a 10 to 90% acetonitrile gradient containing 0.1% TFA. (B) Lesion length measurements of XA21 rice leaves pretreated with RP-HPLC fractions followed by inoculation with PXO99ΔT. Lesion lengths were measured 12 days after PXO99ΔT inoculation. Each value is the mean±SD from nine inoculated leaves. (C) Deduced amino acid sequence of Ax21 (SEQ ID NO:5). The two peptides (boxed) identified from the biologically active fraction were sequenced using LC-MSMS. Predicted sulfated tyrosines Y22 and Y144 are underlined. The dashed box indicates one of the peptide used in the Ax21 bioassay shown in FIG. 3. (D) Mass (LTQ) spectrum of the axY22 peptide corresponding to the N-terminal region (first box in FIG. 1C) of Ax21. The spectrum corresponding to the peptide derived from the C-terminal region (second box in FIG. 1C) of Ax21 is shown in FIG. 51.

FIG. 2. A mutation in ax2l abolishes Ax21 activity. (A) Lesion lengths of rice leaves measured 12 days after inoculation with Xoo strains PXO99, PXO99ΔraxST or PXO99Δax21. Suspensions of each strain (1×10⁸ CFU/mL) were scissor-inoculated onto rice leave (TP309-XA21; resistant to PXO99 and TP309; susceptible to PXO99). The experiment shown here is representative of 5 independent experiments. (B) Growth of PXO99, PXO99ΔraxST and PXO99Aax21 populations in inoculated rice leaves. Bacteria were extracted from the leaves at 0, 3, 6, 9, and 12 days after inoculation, plated on selective media after serial dilution, and colonies counted after a three-day incubation at 28° C. Each value is the mean ±SD from nine inoculated leaves.

FIG. 3. The AxY^{S}22 peptide is sufficient to trigger Xa21-mediated immunity. (A)

Synthetic peptides, including three corresponding to the N-terminal region of AX21 (axY^{S}22 (AENLS(sulfated Y)NFVEGDYVRTP; SEQ ID NO:1), axY22 (AENLSYNFVEGDYVRTP; SEQ ID NO:4), and axY22A (AENLSANFVEGDYVRTP; SEQ ID NO:6)), three corresponding to the central region (axY^{S}144 (YALAGYED(sulfated Y)SKKRGIDA; SEQ ID NO:7), axY144 (YALAGYEDYSKKRGIDA; SEQ ID NO:8), and axY144A (YALAGYEDASKKRGIDA; SEQ ID NO:9)), and one corresponding to the C-terminal region (axM178 (MDGDGNKEW ; SEQ ID NO:10) were tested for activity. (B) Five hours after peptide pretreatment, leaves were inoculated with PXO99ΔraxST and the lesions measured 12 days later. Each value is the mean ±SD from 6 leaves. (C) Growth of PXO99ΔraxST populations over time. TP309-XA21 leaves were pretreated with PXO99 supernatant (PXO99sup), water, or 100 μM of the synthetic peptides (axY^{S}22 and axY22). Bacterial cells were extracted from the leaves at 0, 5,10 and 15 days after inoculation, plated on selective media after serial dilution, and colonies counted after a three-day incubation at 28° C. Each value is the mean±SD from 8 inoculated leaves.

FIG. 4. XA21 is required for AxY^{S}22 binding. HA tagged AxY^{S}22 cross-links to a 140 kDa polypeptide that is immunoprecipitated by an anti-Myc Antibody (Myc-XA21). (A) Before immunoprecipitation, the loading of equal amounts of protein (50 μg) from Kitaake and Myc-XA21 leaf extracts was confirmed using an anti-actin antibody (input). (B) Leaf extracts were incubated with 1 mM of HA-AxY^{S}22 in the presence (+: 5 mM, ++: 10 mM) or absence (−) of the competitors AxY^{S}22 lacking the HA tag or flg22ave. After binding, cross-linking was initiated by the addition of sulfo-EGS. Duplicate protein gels were analyzed after separation by SDS-PAGE using anti-Myc (upper) and anti-HA (lower) antibodies. Myc-XA21 and a proteolytic cleavage product of Myc-XA21 were detected at 140 and 110 kDa, respectively, as reported previously (C. J. Park et al., PLoS Biol 6, e231 (2008)). Arrows indicate the XA21 and Ax21/XA21 complexes.

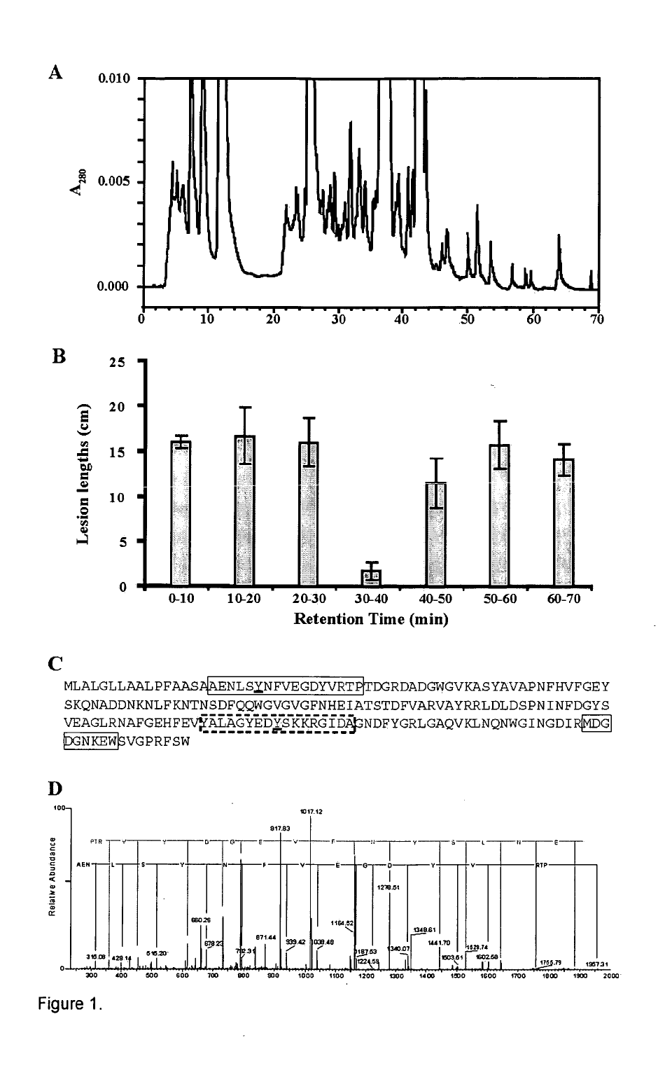

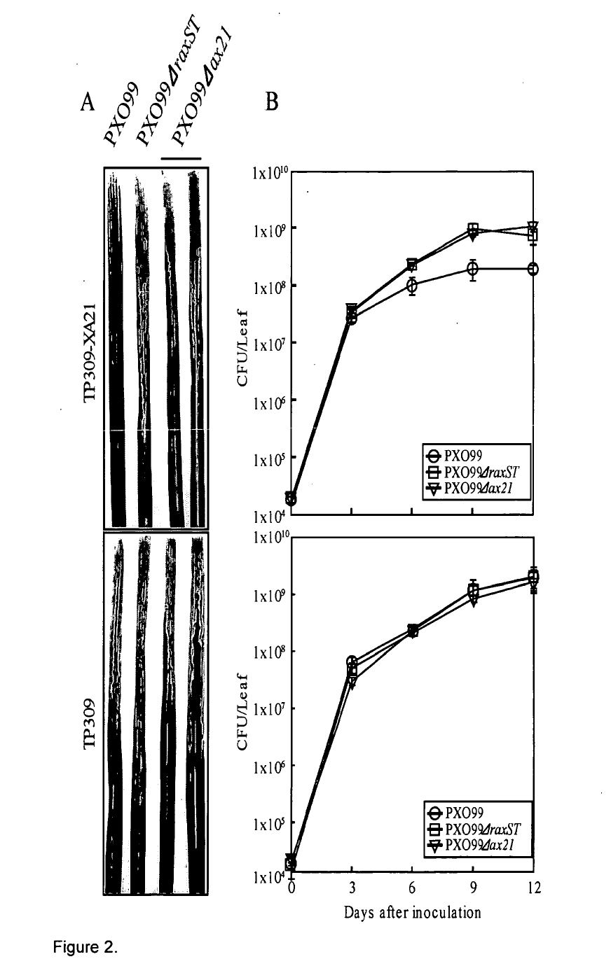

FIG. 5. Identification of the Ax21 protein using LC-MS/MS. (A) Deduced amino acid sequence of Ax21 (SEQ ID NO:5). The two peptides (boxes) identified from the biologically active fraction 4 isolated using RP-HPLC (see FIG. 1) were sequenced using LC-MSMS. The predicted sulfated tyrosines Y22 and Y144 are underlined. Dashed box indicates the peptide synthesized for the Ax21 activity bioassay. (B) Mass (LTQ) spectrum of the peptide corresponding to the C-terminal region (the second box) of the Ax21 protein (SEQ ID NO:10). The spectrum corresponding to the N-terminal region (the first box) of Ax21 is shown in FIG. 2.

FIG. 6. Lesion length analysis of Xoo strains carrying knockouts in eight candidate genes identified through LC-MSMS of the AX21-active fraction. Six-week old rice leaves carrying XA21 were inoculated with mutants carrying deletions for each of the Ax21 candidate genes using the scissors clipping method and then lesion length were measured after two weeks. Each value is the mean±SD from more than 10 inoculated leaves. This experiment shown here is representative of two independent experiments.

FIG. 7. Ax21 is secreted from PXO99 but not from the mutant strains PXO99ΔraxA and PXO99ΔraxC. (A) SDS-PAGE analysis showing peptides extracted from PXO99, PXO99ΔraxA, and PXO99ΔraxC supernatants. The arrow indicates the 20 kD band present in the PXO99 supernatant. This band is absent in the supernatants collected from PXO99ΔraxA and PXO99ΔraxC strains. (B) Following in gel digestion with trypsin of the 20 kD band, LC-MS analysis was carried out to identify the corresponding proteins. 7 of peptide fragments were revealed, all corresponding to Ax21 (SEQ ID NO:11) and covering 68% of the protein.

FIG. 8. Alanine scanning mutagenesis of the AxY^{S}22 peptide. (A) N19, S21, Y22, V25, E26, G27, R31 (partially) and T32 are critical for Ax21 activity. Seventeen AxY^{S}22 peptide variants (SEQ ID NOS:12-28 carrying alanine substitutions were tested for Ax21 activity. TP309-XA21 leaves were pretreated with 100 μM of each peptide solution was and then inoculated five hours later with PXO99ΔraxST. Lesion lengths were measured and 18 days later. Each value is the mean±SD from seven inoculated leaves. (B) A concentration of 1 μM is sufficient for PAMP activity. TP309-XA21 leaves were pretreated with different concentrations (50, 10, 1, or 0.1 μM) of AxY^{S}22 peptide and then lesion development by PXO99ΔraxST was measured 21 days after inoculation. Each value is the mean±SD from seven inoculated leaves.

FIG. 9. Ax21 is highly conserved in all sequenced Xanthomonas strains. (A) Amino acid sequence alignment with putative Ax21 orthologs from ten Xanthomonas species, Xylella fastidiosa (Xf), and a human pathogen, S. maltophilia (Sm) using ClutalW2 (http://www.ebi.ac.uk/Tools/clustalw2/index.html). (B) Phylogenetic tree showing putative Ax21 orthologs from four strains pathogenic on rice (Oryza sativa) [Xoo KACC 10331 (Xoo KACC), Xoo 311018 (Xoo MAFF), Xoo PXO99, and X. oryzae pv. oryzicola BLS256 (Xoc)], a strain pathogenic on citrus (X. axonopodis pv. citri 306, Xac), a strain pathogenic on tomato and pepper (X. axonopodis pv. vesicatoria 85-10, Xav), a strain pathogenic on soybean (X. axonopodis pv. glycines 8ra, Xag), strains pathogenic on Brassica and Arabidopsis [X. campestris pv. campestris 33919 (Xcc 33919), 8004 (Xcc 8004), and B100 (Xcc B 100)], a fastidious bacterial strain X. fastidiosa Dixon [a causal agent of several plant diseases (phoney peach disease, oleander leaf scorch and Pierce's disease, and citrus X disease)], a strain that is pathogenic on humans (Stenotrophomonas maltophilia, Sm), strains that are ocean bacteria [Pseudoalteromonas atlantica (Pa), Alteromonas macleodi (Am), and Idiomarina loihiensis (Il)], and a strain that is a symbiotic green sulfur bacterium (Chlorobium chlorochromatii, Cc). The strain number, genome accession number and GenBank accession number for each putative ortholog are as follow: X. oryzae pv. oryzae PXO99, CP000967, PXO_{—}03968 (SEQ ID NO:29); X. oryzae pv. oryzae MAFF, AP008229, XOO3968 (SEQ ID NO:31); X. oryzae pv. oryzae KACC, AE013598, XOO4199 (SEQ ID NO:30); X. oryzae pv. oryzicola, AAQN00000000, Xoryp_{—}01570(SEQ ID NO:32); X. axonopodis pv. citri, AE008923, XACO223 (SEQ ID NO:34); X. axonopodis pv. vesicatoria, AM039952, XCV0208 (SEQ ID NO:33); X. axonopodis pv. glycines, AAS91338 (SEQ ID NO:38); X. campestris pv. campestris 33913, AE008922 (SEQ ID NO:36); X. campestris pv. campestris 8004, CP000050, XCC0205 (SEQ ID NO:35); X. campestris pv. campestris B100, AM920689, XCCB100_{—}0226 (SEQ ID NO:37); X. fastidiosa, AAAL00000000, XfasaDRAFT_{—}1077 (SEQ ID NO:39); S. maltophilia, AM743169, Smlt0387 (SEQ ID NO:40); P. atlantica, ABG38916, Patl_{—}0386 (SEQ ID NO:41); A. macleodi, ACG68266, MADE_{—}03976 (SEQ ID NO:42); I. loihiensis L2TR, AAV82257, IL1417 (SEQ ID NO:43); C. chlorochromatii CaD3; ABB27819, Cag_{—}0546 (SEQ ID NO:44). Consensus=SEQ ID NO:45)

FIG. 10. Ax21 activity in X. axonopodis pv. vesicatoria. The supernatants of Xoo strain PXO99, PXO99ΔraxST, Xav strain 8510, and XavAax21 were used to pretreat XA21 and TP309 rice leaves as described in materials and methods. After pretreatment, the rice leaves were inoculated with Xoo mutant strain PXO99ΔraxST. Lesion lengths were measured three weeks after PXO99ΔraxST inoculation. Each bar indicates the average lesion length±SD from 7 or 9 leaves.

### DETAILED DESCRIPTION OF THE INVENTION

### I. Introduction

The present invention is based, in part, on the discovery that XA21-based disease resistance is triggered in part by plant recognition of a sulfated peptide sequence from X. oryzae. As shown in the examples, contacting a plant with this peptide is sufficient to induce disease resistance in plants. Accordingly, the present invention provides for purified sulfated peptides, compositions comprising such peptides, and methods of making and using such peptides for inducing or increasing disease resistance.

### II. Polypeptides of the Invention

The present invention provides polypeptides that induce or enhance disease resistance in a plant. As described in the Examples, the inventors have discovered that a sulfated peptide plays a role in pathogen recognition and disease resistance in plants. Specifically, the inventors have found that AENLSY*NFVEGDYVRTP (SEQ ID NO:1), wherein Y* represents a sulfated (or optionally, phosphorylated) tyrosine, is sufficient to induce XA21-mediated disease resistance.

While the inventors have identified activity in a particular peptide, it will be appreciated that variants of that peptide can also be used to induce or enhance disease resistance. In some embodiments, the polypeptides of the invention comprise one or more (e.g., 1, 2, 3, 4, 5 or more) amino acid insertions, deletions or modifications (e.g., substitution of one amino acid for another) compared to SEQ ID NO:1 or are otherwise substantially identical (e.g., having a sequence at least 80%, 85%, 90%, 95%, 98%, or more identical with the entire sequence of SEQ ID NO:1). For example, polypeptides comprising or consisting of an amino acid sequence having one or more (e.g., 1, 2, 3, 4, 5, or more) conservative amino acid substitutions relative to SEQ ID NO:1 (but retaining the sulfated (or optionally, phosphorylated) tyrosine, Y*) are in polypeptides of the invention. Moreover, as shown in FIG. 9, other bacteria (e.g., Stenotrophomonas and Xylella species) also carry variants of SEQ ID NO:1. Polypeptides comprising or consisting of these sequences are provided wherein the relevant tyrosine (e.g., as determined by alignment with SEQ ID NO:1). is sulfated (or optionally, phosphorylated). Alignment of SEQ ID NO:1 with amino acid sequences from these other bacteria allows for identification of positions that support disclosure of active peptide variants. Accordingly, in some embodiments, a polypeptide of the invention comprises or consists of A(E/Q)(N/G)LSY*N(F/Y)(V/A)(E/G)(G/A/S)DY(V/A)(R/K)T(P/D/K) (SEQ ID NO:2), wherein Y* represents a sulfated (or optionally, phosphorylated) tyrosine.

Moreover, the polypeptides of the invention include active fragments of the above-described polypeptides. In some embodiments, active fragments comprise at least the fragment LSY*N, and optionally comprise at least 1, 2, 3, 4, 5, 6, 7, or more contiguous amino acids of SEQ ID NO:1 or SEQ ID NO:2. In some embodiments, the polypeptides of the invention comprise or consist of SEQ ID NO:1 or a variant thereof as described above, but lacks 1, 2, 3, or more of the N and/or C-terminal amino acids as set forth in SEQ ID NO:1 or SEQ ID NO:2, wherein the polypeptide retains the sulfated (or optionally, phosphorylated) tyrosine (Y*). Thus, for example, in some embodiments, a polypeptide of the invention comprises or consists of, e.g.,

(SEQ ID NO: 46)
(E/Q)(N/G)LSY*N(F/Y)(V/A)(E/G)(G/A/S)DY(V/A)(R/K)
T(P/D/K),
(SEQ ID NO: 47)
(N/G)LSY*N(F/Y)(V/A)(E/G)(G/A/S)DY(V/A)(R/K)T(P/
D/K),
(SEQ ID NO: 48)
A(E/Q)(N/G)LSY*N(F/Y)(V/A)(E/G)(G/A/S)DY(V/A)(R/
K)T,
(SEQ ID NO: 49)
A(E/Q)(N/G)LSY*N(F/Y)(V/A)(E/G)(G/A/S)DY(V/A)(R/
K),
(SEQ ID NO: 50)
ENLSY*NFVEGDYVRTP
(SEQ ID NO: 51)
NLSY*NFVEGDYVRTP
(SEQ ID NO: 52)
AENLSY*NFVEGDYVRT
(SEQ ID NO: 53)
AENLSY*NFVEGDYVR.

The sequences described herein can be the sole amino acids of a polypeptide of the invention or additional amino acids at one or both ends of the polypeptide. For example, in some embodiments, a polypeptide of the present invention will be a fusion protein comprising one or more additional polypeptide sequences. Such sequence can include, but are not limited to, polypeptide sequences with other biological activities (e.g., other avirulence gene products or elicitors, or inducing other desirable traits in a plant) and/or polypeptide sequences useful for monitoring the polypeptide (e.g., tags or other sequences), protease or other cleavable sequences, additional pro-domains (e.g., domains that obscure the active peptide domain until the pro-domain is cleaved), etc. In some embodiments, the polypeptides of the invention comprise additional portions or the entire avrXA21 amino acid sequence, or conservative variants thereof that retain activity. Exemplary avrXA21 full-length sequences are provided, for example, in FIG. 9.

The polypeptides of the invention can be part of an organism (e.g., expressed in a cell of the organism) or isolated cell, or can be purified and/or isolated from one or more components of a cell. Isolated polypeptides can also be isolated can also be generated by peptide synthesis. Those of skill in the art will recognize that polypeptides can be generated by synthetic or recombinant methods. In some embodiments, the invention provides for isolated or purified cells or cell cultures that express a polypeptide of the invention. Such cells (e.g., recombinantly engineered to express a polypeptide of the invention) can be any type of cell. Exemplary expression systems include various bacterial, fungal and yeast, insect, plant, and mammalian expression systems.

Optionally, the cells (or organisms comprising such cells, e.g., plants) expressing the polypeptides of the invention include one or more additional proteins that add a sulfate moiety to the Y* tyrosine as described herein. In some cases, for example, the cells further express the raxST gene, or an ortholog or other active variant thereof, thereby resulting in sulfation of the Y* tyrosine. For example, in some embodiments, a host cell (e.g., a bacterial cell, e.g., a Xanthamonas cell, e.g., a Xoo cell) is modified to express a RaxST sulfotransferase and to express an avrXA21 polypeptide as described herein. The avrXA21 polypeptide is then sulfated in the cell and can be purified. Optionally, the expressed avrXA21 polypeptide is a fusion protein wherein a tag is fused to avrXA21. The fusion protein can then be purified based on the presence of the tag.

Alternatively, the polypeptides of the invention can be generated by cells, wherein the tyrosine at the Y* position has not been sulfated. In these cases, the polypeptide can be sulfated by either chemical or enzymatic methods following production, and optionally following at least partial purification of the polypeptide from a cell or cell mixture. In some embodiments, a sulfotransferase (e.g., RaxST) is expressed in another cell (e.g., E. coli), optionally purified, and contacted to a purified axrXA21 polypeptide under conditions to allow for sulfation of the avrXA21 polypeptide.

### III. Methods of Using the Polypeptides of the Invention

The polypeptides of the present invention have a number of uses. Notably, contacting a plant with the polypeptides of the invention is capable of inducing disease resistance in a plant. The contacting can occur by applying exogenous (i.e., not expressed in the plant) polypeptide to the plant or the polypeptide can be expressed in the plant.

In some embodiments, the plants express XA21 or a functional equivalent, i.e., a disease resistance gene product that enables recognition of a polypeptide of the invention and subsequent induction of disease resistance. The XA21 gene was first isolated from rice. See, e.g., Tunkel, C. et al., Mol. Microbiol. 58:289 (2005); Song, W. et al., Science 270:1804 (1995). See also U.S. Pat. No. 5,977,434. The plant can express XA21 endogenously (e.g., not by recombinant expression) or the plant can be transgenic or otherwise recombinantly manipulated to express XA21. Thus, while XA21 was originally identified in rice, XA21 can be expressed in plants other than rice. Moreover, it is believed that plants (other than rice plants) can be identified that have XA21-activity, i.e., the plants have enhanced disease resistance in response to contact with a polypeptide of the invention. Indeed, the present invention provides for methods of identifying such plants by contacting a plurality of non-rice plants (e.g., of diverse genetic background) with a polypeptide of the invention (either purified or expressed from a bacterial or fungal pathogen) and identifying a contacted plant that has enhanced disease resistance or other manifestation of disease resistance (e.g., a hypersensitive response) as a result the presence of the polypeptide in the contacting step.

It is believed that one mode of action of the avrXA21 polypeptides of the invention is to act as bacterial quorum (QS) sensors. QS is used by a number of bacterial species, including bacterial causal agents of animal and human disease. It is believed that administration of a therapeutically effective amount of an avrXA21 polypeptide of the invention, and especially a dominant-negative variant of the polypeptide, will disrupt QS functions of bacterial animal or human pathogens in animals (e.g., bovines, poultry animals, pigs, sheep, dogs, cats, horses, rats, mice, etc.) or humans, respectively, thereby treating or ameliorating disease caused by such bacterial agents. Variants of the avrXA21 polypeptides that block endogenous bacterial QS peptides are of particular interest. Such treatment is expected to be effective against any bacterial animal or human pathogen that uses a polypeptide substantially identical to an avrXA21 polypeptide of the invention as a QS agent. Exemplary bacterial species include, but are not limited to, Staphylococcus aureus, Bordetella pertussis, Stenotrophomonas maltophilia, Lactobacillus plantarum and Lactobacillus sake.

Administration of avrXA21 polypeptides described herein can be by any of the routes normally used for introducing pharmaceuticals. The pharmaceutical compositions of the invention may comprise a pharmaceutically acceptable carrier. Pharmaceutically acceptable carriers are determined in part by the particular composition being administered, as well as by the particular method used to administer the composition. Accordingly, there are a wide variety of suitable formulations of pharmaceutical compositions of the present invention (see, e.g., Remington's Pharmaceutical Sciences, 17^{th} ed. 1985)).

Formulations suitable for administration include aqueous and non-aqueous solutions, isotonic sterile solutions, which can contain antioxidants, buffers, bacteriostats, and solutes that render the formulation isotonic, and aqueous and non-aqueous sterile suspensions that can include suspending agents, solubilizers, thickening agents, stabilizers, and preservatives. In the practice of this invention, compositions can be administered, for example, orally, nasally, topically, intravenously, intraperitoneally, intrathecally or into the eye (e.g., by eye drop or injection). The formulations of compounds can be presented in unit-dose or multi-dose sealed containers, such as ampoules and vials. Solutions and suspensions can be prepared from sterile powders, granules, and tablets of the kind previously described. The modulators can also be administered as part of a prepared food or drug.

The dose administered to a patient, in the context of the present invention should be sufficient to induce a beneficial response in the subject over time, i.e., to ameliorate a condition of the subject. The optimal dose level for any patient will depend on a variety of factors including the efficacy of the specific modulator employed, the age, body weight, physical activity, and diet of the patient, and on a possible combination with other drug. The size of the dose also will be determined by the existence, nature, and extent of any adverse side-effects that accompany the administration of a particular compound or vector in a particular subject. Administration can be accomplished via single or divided doses.

### IV. Agricultural Formulations

The present invention provides for agricultural formulations formulated for contacting to plants, wherein the formulation comprises a polypeptide (e.g., a sulfated polypeptide as described herein) of the present invention. The formulations can be suitable for treating plants or plant propagation material, such as seeds, in accordance with the present invention, e.g., in a carrier. Suitable additives include buffering agents, wetting agents, coating agents, polysaccharides, and abrading agents. Exemplary carriers include water, aqueous solutions, slurries, solids and dry powders (e.g., peat, wheat, bran, vermiculite, clay, pasteurized soil, many forms of calcium carbonate, dolomite, various grades of gypsum, bentonite and other clay minerals, rock phosphates and other phosphorous compounds, titanium dioxide, humus, talc, alginate and activated charcoal). Any agriculturally suitable carrier known to one skilled in the art would be acceptable and is contemplated for use in the present invention. Optionally, the formulations can also include at least one surfactant, herbicide, fungicide, pesticide, or fertilizer.

Treatment can be performed using a variety of known methods, e.g., by spraying, atomizing, dusting or scattering the compositions over the propagation material or brushing or pouring or otherwise contacting the compositions over the plant or, in the event of seed, by coating, encapsulating, or otherwise treating the seed. In an alternative to directly treating a plant or seed before planting, the formulations of the invention can also be introduced into the soil or other media into which the seed is to be planted. In some embodiments, a carrier is also used in this embodiment. The carrier can be solid or liquid, as noted above. In some embodiments peat is suspended in water as a carrier of the polypeptide of the invention, and this mixture is sprayed into the soil or planting media and/or over the seed as it is planted.

### V. Preparation of Recombinant Vectors

To use isolated sequences in the above techniques, recombinant DNA vectors suitable for transformation of plant cells are prepared. Techniques for transforming a wide variety of higher plant species are well known and described in the technical and scientific literature, e.g., Weising et al. Ann. Rev. Genet. 22:421-477 (1988). A DNA sequence coding for the desired polypeptide (e.g., a polypeptide as described herein, including but not limited to a polypeptide comprising SEQ ID NO:1 or 2), will be combined with transcriptional and translational initiation regulatory sequences which will direct the transcription of the sequence from the gene in the intended tissues of the transformed plant.

For example, for overexpression, a plant promoter fragment may be employed which will direct expression of the gene in all tissues of a regenerated plant. Alternatively, the plant promoter may direct expression of the polynucleotide of the invention in a specific tissue (tissue-specific promoters), organ (organ-specific promoters) or may be otherwise under more precise environmental control (inducible promoters). Examples of tissue-specific promoters under developmental control include promoters that initiate transcription only in certain tissues, such as fruit, seeds, flowers, pistils, or anthers. Suitable promoters include those from genes encoding storage proteins or the lipid body membrane protein, oleosin.

If proper polypeptide expression is desired, a polyadenylation region at the 3′-end of the coding region should be included. The polyadenylation region can be derived from the natural gene, from a variety of other plant genes, or from T-DNA.

The vector comprising the sequences (e.g., promoters or coding regions) from genes of the invention will typically comprise a marker gene that confers a selectable phenotype on plant cells. For example, the marker may encode biocide resistance, particularly antibiotic resistance, such as resistance to kanamycin, G418, bleomycin, hygromycin, or herbicide resistance, such as resistance to chlorosulfuron or Basta.

Constitutive Promoters

A promoter, or an active fragment thereof, can be employed which will direct expression of a nucleic acid encoding a fusion protein of the invention, in all transformed cells or tissues, e.g., as those of a regenerated plant. Such promoters are referred to herein as “constitutive” promoters and are active under most environmental conditions and states of development or cell differentiation. Examples of constitutive promoters include those from viruses which infect plants, such as the cauliflower mosaic virus (CaMV) 35S transcription initiation region (see, e.g., Dagless Arch. Virol. 142:183-191 (1997)); the 1′- or 2′-promoter derived from T-DNA of Agrobacterium tumefaciens (see, e.g., Mengiste supra (1997); O'Grady Plant Mol. Biol. 29:99-108) (1995)); the promoter of the tobacco mosaic virus; the promoter of Figwort mosaic virus (see, e.g., Maiti Transgenic Res. 6:143-156) (1997)); actin promoters, such as the Arabidopsis actin gene promoter (see, e.g., Huang Plant Mol. Biol. 33:125-139 (1997)); alcohol dehydrogenase (Adh) gene promoters (see, e.g., Millar Plant Mol. Biol. 31:897-904 (1996)); ACT11 from Arabidopsis (Huang et al. Plant Mol. Biol. 33:125-139 (1996)), Cat3 from Arabidopsis (GenBank No. U43147, Zhong et al., Mol. Gen. Genet. 251:196-203 (1996)), the gene encoding stearoyl-acyl carrier protein desaturase from Brassica napus (Genbank No. X74782, Solocombe et al. Plant Physiol. 104:1167-1176 (1994)), GPc1 from maize (GenBank No. X15596, Martinez et al. J. Mol. Biol 208:551-565 (1989)), Gpc2 from maize (GenBank No. U45855, Manjunath et al., Plant Mol. Biol. 33:97-112 (1997)), other transcription initiation regions from various plant genes known to those of skill. See also Holtorf (1995) “Comparison of different constitutive and inducible promoters for the overexpression of transgenes in Arabidopsis thaliana,” Plant Mol. Biol. 29:637-646.

Inducible Promoters

Alternatively, a plant promoter may direct expression of the nucleic acids under the influence of changing environmental conditions or developmental conditions. Examples of environmental conditions that may effect transcription by inducible promoters include anaerobic conditions, elevated temperature, drought, or the presence of light. Example of developmental conditions that may effect transcription by inducible promoters include senescence and embryogenesis. Such promoters are referred to herein as “inducible” promoters.

Exemplary inducible promoters include those promoters that are specifically induced upon infection by a virulent pathogen. Selected promoters useful in the invention are discussed in PCT application WO 99/43824, and include promoters from:
a. lipoxygenases (e.g., Peng et al, J. Biol. Chem. 269:3755-3761 (1994)),
b. peroxidases (e.g., Chittoor et al. Molec. Plant-Microbe Interact. 10:861-871 (1997)),
c. hydroxymethylglutaryl-CoA reductase,
d. phenylalanine ammonia lyase,
e. glutathione-S-transferase,
f. chitinases (e.g., Zhu et al. Mol. Gen. Genet. 226:289-296 (1991)),
g. genes involved in the plant respiratory burst (e.g., Groom et al. Plant J. 10(3):515-522 (1996)); and
h. pathogenesis-related (PR) protein promoters.

Other examples of developmental conditions include cell aging, and embryogenesis. For example, the invention incorporates the senescence inducible promoter of Arabidopsis, SAG 12, (Gan and Amasino, Science, 270:1986-1988 (1995)) and the embryogenesis related promoters of LEC1 (Lotan et al., Cell, 93:1195-205 (1998)), LEC2 (Stone et al., Proc. Natl. Acad. of Sci., 98:11806-11811 (2001)), FUS3 (Luerssen, Plant J. 15:755-764 (1998)), AtSERK1 (Hecht et al. Plant Physiol 127:803-816 (2001)), AGL15 (Heck et al. Plant Cell 7:1271-1282 (1995)), and BBM (BABYBOOM). Other inducible promoters include, e.g., the drought-inducible promoter of maize (Busk supra (1997)) and the cold, drought, and high salt inducible promoter from potato (Kirch Plant Mol. Biol. 33:897-909 (1997)).

Alternatively, plant promoters which are inducible upon exposure to plant hormones, such as auxins or cytokinins, are used to express the nucleic acids of the invention. For example, the invention can use the auxin-response elements E1 promoter fragment (AuxREs) in the soybean (Glycine max L.) (Liu Plant Physiol. 115:397-407 (1997)); the auxin-responsive Arabidopsis GST6 promoter (also responsive to salicylic acid and hydrogen peroxide) (Chen Plant J. 10:955-966 (1996)); the auxin-inducible parC promoter from tobacco (Sakai 37:906-913 (1996)); a plant biotin response element (Streit Mol. Plant Microbe Interact. 10:933-937 (1997)); and, the promoter responsive to the stress hormone abscisic acid (Sheen Science 274:1900-1902 (1996)). The invention can also use the cytokinin inducible promoters of ARR5 (Brandstatter and Kieber, Plant Cell, 10:1009-1019 (1998)), ARR6 (Brandstatter and Kieber, Plant Cell, 10:1009-1019 (1998)), ARR2 (Hwang and Sheen, Nature, 413:383-389 (2001)), the ethylene responsive promoter of ERF1 (Solano et al., Genes Dev. 12:3703-3714 (1998)), and the β-estradiol inducible promoter of XVE (Zuo et al., Plant J, 24:265-273 (2000)).

Plant promoters which are inducible upon exposure to chemicals reagents which can be applied to the plant, such as herbicides or antibiotics, are also used to express the nucleic acids of the invention. For example, the maize In2-2 promoter, activated by benzenesulfonamide herbicide safeners, can be used (De Veylder Plant Cell Physiol. 38:568-577 (1997)) as well as the promoter of the glucocorticoid receptor protein fusion inducible by dexamethasone application (Aoyama, Plant J., 11:605-612 (1997)); application of different herbicide safeners induces distinct gene expression patterns, including expression in the root, hydathodes, and the shoot apical meristem. The coding sequence of the described nucleic acids can also be under the control of, e.g., a tetracycline-inducible promoter, e.g., as described with transgenic tobacco plants containing the Avena sativa L. (oat) arginine decarboxylase gene (Masgrau Plant J. 11:465-473 (1997)); or, a salicylic acid-responsive element (Stange Plant J. 11:1315-1324 (1997)).

Tissue-Specific Promoters

Examples of tissue-specific promoters under developmental control include promoters that initiate transcription only (or primarily only) in certain tissues, such as vegetative tissues, e.g., roots, leaves or stems, or reproductive tissues, such as fruit, ovules, seeds, pollen, pistils, flowers, or any embryonic tissue.

A variety of promoters specifically active in vegetative tissues, such as leaves, stems, roots and tubers, can also be used to express the nucleic acids used in the methods of the invention. For example, promoters controlling patatin, the major storage protein of the potato tuber, can be used, e.g., Kim Plant Mol. Biol. 26:603-615 (1994); Martin Plant J. 11:53-62 (1997). The ORF13 promoter from Agrobacterium rhizogenes which exhibits high activity in roots can also be used (Hansen Mol. Gen. Genet. 254:337-343 (1997)). Other useful vegetative tissue-specific promoters include: the tarin promoter of the gene encoding a globulin from a major taro (Colocasia esculenta L. Schott) corm protein family, tarin (Bezerra Plant Mol. Biol. 28:137-144 (1995)); the curculin promoter active during taro corm development (de Castro Plant Cell 4:1549-1559 (1992)) and the promoter for the tobacco root-specific gene TobRB7, whose expression is localized to root meristem and immature central cylinder regions (Yamamoto Plant Cell 3:371-382 (1991)).

Leaf-specific promoters, such as the ribulose biphosphate carboxylase (RBCS) promoters can be used. For example, the tomato RBCS1, RBCS2 and RBCS3A genes are expressed in leaves and light-grown seedlings, only RBCS1 and RBCS2 are expressed in developing tomato fruits (Meier FEBS Lett. 415:91-95 (1997)). A ribulose bisphosphate carboxylase promoters expressed almost exclusively in mesophyll cells in leaf blades and leaf sheaths at high levels, described by Matsuoka Plant J. 6:311-319 (1994), can be used. Another leaf-specific promoter is the light harvesting chlorophyll a/b binding protein gene promoter, see, e.g., Shiina Plant Physiol. 115:477-483 (1997); Casal Plant Physiol. 116:1533-1538 (1998). The Arabidopsis thaliana myb-related gene promoter (Atmyb5) described by Li FEBS Lett. 379:117-121 (1996), is leaf-specific. The Atmyb5 promoter is expressed in developing leaf trichomes, stipules, and epidermal cells on the margins of young rosette and cauline leaves, and in immature seeds. Atmyb5 mRNA appears between fertilization and the 16-cell stage of embryo development and persists beyond the heart stage. A leaf promoter identified in maize by Busk Plant J. 11:1285-1295 (1997), can also be used.

Another class of useful vegetative tissue-specific promoters are meristematic (root tip and shoot apex) promoters. For example, the “SHOOTMERISTEMLESS” and “SCARECROW” promoters, which are active in the developing shoot or root apical meristems, described by Di Laurenzio Cell 86:423-433 (1996) and Long Nature 379:66-69 (1996), can be used. Another useful promoter is that which controls the expression of 3-hydroxy-3-methylglutaryl coenzyme A reductase HMG2 gene, whose expression is restricted to meristematic and floral (secretory zone of the stigma, mature pollen grains, gynoecium vascular tissue, and fertilized ovules) tissues (see, e.g., Enjuto Plant Cell. 7:517-527 (1995)). Also useful are knl-related genes from maize and other species which show meristem-specific expression, see, e.g., Granger Plant Mol. Biol. 31:373-378 (1996); Kerstetter Plant Cell 6:1877-1887 (1994); Hake Philos. Trans. R. Soc. Lond. B. Biol. Sci. 350:45-51 (1995). For example, the Arabidopsis thaliana KNAT1 or KNAT2 promoters. In the shoot apex, KNAT1 transcript is localized primarily to the shoot apical meristem; the expression of KNAT1 in the shoot meristem decreases during the floral transition and is restricted to the cortex of the inflorescence stem (see, e.g., Lincoln Plant Cell 6:1859-1876 (1994)).

One of skill will recognize that a tissue-specific promoter may drive expression of operably linked sequences in tissues other than the target tissue. Thus, as used herein a tissue-specific promoter is one that drives expression preferentially in the target tissue, but may also lead to some expression in other tissues as well.

In another embodiment, a nucleic acid described in the present invention is expressed through a transposable element. This allows for constitutive, yet periodic and infrequent expression of the constitutively active polypeptide. The invention also provides for use of tissue-specific promoters derived from viruses which can include, e.g., the tobamovirus subgenomic promoter (Kumagai Proc. Natl. Acad. Sci. USA 92:1679-1683 (1995)) the rice tungro bacilliform virus (RTBV), which replicates only in phloem cells in infected rice plants, with its promoter which drives strong phloem-specific reporter gene expression; the cassava vein mosaic virus (CVMV) promoter, with highest activity in vascular elements, in leaf mesophyll cells, and in root tips (Verdaguer Plant Mol. Biol. 31:1129-1139 (1996)).

### VI. Production of Transgenic Plants

As discussed above, in some embodiment, the plant comprises:

a first heterologous expression cassette comprising a first promoter operably linked to a polynucleotide encoding a polypeptide of the invention (e.g., comprising a sequence substantially identical to SEQ ID NO:1 or 2, or an active fragment thereof). In some embodiments, such plants endogenously express XA21.

Optionally (e.g., when the plant does not endogenously express XA21 or have endogenous avrXA21-recognition activity), the plant can also comprise, e.g.:

a second heterologous expression cassette comprising a second promoter operably linked to a polynucleotide encoding an XA21 polypeptide (e.g., a polypeptide substantially identical to the XA21 polypeptide described in U.S. Pat. No. 5,977,434); and/or

a third heterologous expression cassette comprising a third promoter operably linked to a polynucleotide encoding a sulfur transferase (e.g., raxST gene, or an ortholog or other active variant thereof) capable of sulfating the tyrosine Y* of the polypeptide product of the first expression cassette.

Accordingly, the present invention provides for transgenic plants comprising the first and second expression cassettes, comprising the first and third expression cassettes or all three expression cassettes.

In some embodiments, the invention provides for a transgenic plant comprising the second expression cassette contacted with an exogenous polypeptide of the invention (e.g., comprising a sequence substantially identical to SEQ ID NO:1 or 2, or an active fragment thereof).

DNA constructs of the invention may be introduced into the genome of the desired plant host by a variety of conventional techniques. For example, the DNA constructs may be introduced directly into the genomic DNA of the plant cell using techniques such as electroporation and microinjection of plant cell protoplasts, or the DNA constructs can be introduced directly to plant tissue using biolistic methods, such as DNA particle bombardment. Alternatively, the DNA constructs may be combined with suitable T-DNA flanking regions and introduced into a conventional Agrobacterium tumefaciens host vector. The virulence functions of the Agrobacterium tumefaciens host will direct the insertion of the construct and adjacent marker into the plant cell DNA when the cell is infected by the bacteria.

Microinjection techniques are known in the art and well described in the scientific and patent literature. The introduction of DNA constructs using polyethylene glycol precipitation is described in Paszkowski et al. Embo J. 3:2717-2722 (1984). Electroporation techniques are described in Fromm et al. Proc. Natl. Acad. Sci. USA 82:5824 (1985). Biolistic transformation techniques are described in Klein et al. Nature 327:70-73 (1987).

Agrobacterium tumefaciens-mediated transformation techniques, including disarming and use of binary vectors, are well described in the scientific literature. See, for example Horsch et al,. Science 233:496-498 (1984), and Fraley et al. Proc. Natl. Acad. Sci. USA 80:4803 (1983).

Transformed plant cells which are derived by any of the above transformation techniques can be cultured to regenerate a whole plant which possesses the transformed genotype and thus the desired phenotype such as increased disease resistance compared to a control plant that was not transformed or transformed with an empty vector. Such regeneration techniques rely on manipulation of certain phytohormones in a tissue culture growth medium, typically relying on a biocide and/or herbicide marker which has been introduced together with the desired nucleotide sequences. Plant regeneration from cultured protoplasts is described in Evans et al., Protoplasts Isolation and Culture, Handbook of Plant Cell Culture, pp. 124-176, MacMillilan Publishing Company, New York, 1983; and Binding, Regeneration of Plants, Plant Protoplasts, pp. 21-73, CRC Press, Boca Raton, 1985. Regeneration can also be obtained from plant callus, explants, organs, or parts thereof. Such regeneration techniques are described generally in Klee et al. Ann. Rev. of Plant Phys. 38:467-486 (1987).

The nucleic acids and encoded polypeptides of the invention can be used to confer enhanced disease resistance on essentially any plant. Thus, the invention has use over a broad range of plants, including species from the genera Asparagus, Atropa, Avena, Brassica, Citrus, Citrullus, Capsicum, Cucumis, Cucurbita, Daucus, Fragaria, Glycine, Gossypium, Helianthus, Heterocallis, Hordeum, Hyoscyamus, Lactuca, Linum, Lolium, Lycopersicon, Malus, Manihot, Majorana, Medicago, Nicotiana, Oryza, Panieum, Pannesetum, Persea, Pisum, Pyrus, Prunus, Raphanus, Secale, Senecio, Sinapis, Solanum, Sorghum, Trigonella, Triticum, Vitis, Vigna, and, Zea.

VII. Selecting for Plants with Enhanced Resistance

Plants with enhanced resistance can be selected or indentified in many ways. One of ordinary skill in the art will recognize that the following methods are but a few of the possibilities. One method of selecting plants with enhanced resistance is to determine resistance of a plant to a specific plant pathogen. Possible pathogens include, but are not limited to, viruses, bacteria, nematodes, fungi or insects (see, e.g., Agrios, Plant Pathology (Academic Press, San Diego, Calif.) (1988)). One of skill in the art will recognize that resistance responses of plants vary depending on many factors, including what pathogen or plant is used. Generally, enhanced resistance is measured by the reduction or elimination of disease symptoms when compared to a control plant (e.g., a plant not contacted or expressing a polypeptide of the invention and/or not expressing XA21 or a protein with XA21 activity). In some cases, however, enhanced resistance can also be measured by the production of the hypersensitive response (HR) of the plant (see, e.g., Staskawicz et al. Science 268(5211): 661-7 (1995)). Plants with enhanced resistance can produce an enhanced hypersensitive response relative to control plants.

Enhanced resistance can also be determined by measuring the increased expression of a gene operably linked to a defense-related promoter. Measurement of such expression can be measured by quantifying the accumulation of RNA or subsequent protein product (e.g., using northern or western blot techniques, respectively (see, e.g., Sambrook et al. and

Ausubel et al.). A possible alternate strategy for measuring defense gene promoter expression involves operably linking a reporter gene to the promoter. Reporter gene constructs allow for ease of measurement of expression from the promoter of interest. Examples of reporter genes include: β-gal, GUS (see, e.g., Jefferson, R. A., et al., EMBO J 6:3901-3907 (1987)), green fluorescent protein, luciferase, and others.

### EXAMPLES

In 1995 we showed that the rice Xa21 resistance gene, encoding a protein with predicted leucine rich repeat (LRR), transmembrane, juxtamembrane, and intracellular kinase domains, conferred immunity to diverse strains of the Gram-negative bacterium, Xanthomonas oryzae pv. oryzae (Xoo) (Song, W. et al., Science 270:1804 (1995); Wang, G. L. et al., Mol Plant Microbe Interact 9:850 (1996)). Subsequent discoveries in flies (Toll) (Lemaitre, B. et al., Cell 86:973 (1996)), humans Toll-like receptors 4 (TLR4) (Medzhitov, R. et al., Nature 388:394 (1997)), mice (Poltorak A. et al., Science 282:2085 (1998)), and Arabidopsis (Gomez-Gomez, L. et al., Mol. Cell 5:1003 (2000); Zipfel, C. et al., Cell 125:749 (2006)) revealed that animals and other plant species also carry membrane-anchored receptors with striking structural similarities to XA21 and that these receptors are also involved in microbial recognition and defense. Like XA21, these receptors typically associate with or carry non-RD (arginine-aspartic acid) kinases to control early events of innate immunity signaling (Dardick, C. et al., PLoS pathogens 2:e2 (2006)). Arabidopsis FLS2 and EFR, belong to the same class of plant receptor kinases (the LRRXII) as XA21 (Dardick, C. et al., PLoS pathogens 2:e2 (2006); Shiu, S. H. et al., Plant Cell 16:1220 (2004)).

Many of these cell surface receptors were later named pattern recognition receptors (PRRs) based on their ability to directly recognize molecules that are conserved across a large class of microbes (Medzhitov, R., Nat Rev Immunol 1:135 (2001); Zipfel, C., Curr Opin Plant Biol 12:414 (2009)). Such microbial molecules were called pathogen-associated molecular patterns [PAMPs, also known as microbe associated molecular patterns (MAMPs)] (Medzhitov, R. et al., Curr Opin Immunol 9:4 (1997)).

Despite the similarity of the known PRRs to XA21, the classification of XA21 has been debated (Ausubel, F. M., Nat Immunol 6:973 (2005); Panstruga, R. et al., Cell 136:978 e1 (2009)). This is partly because XA21 was discovered before the terms “PRR” and “PAMP” were established (Medzhitov, R. et al., Curr Opin Immunol 9:4 (1997)) and partly because, under the classical definition of Flor (Flor, H. H., Phytopatholo 32:653 (1942)), XA21 was called a “resistance” gene. Furthermore, because the molecule recognized by XA21 (previously called “avirulenceXa21” (avrXa21) and here renamed Ax21 for Activator of Xa21-mediated immunity) had not been identified, it was not known if this molecule was conserved among a large class of microbes, a hallmark of PAMPs (Medzhitov, R. et al., Curr Opin Immunol 9:4 (1997)).

We previously identified six Xoo genes required for Ax21 activity (rax), which fall into two functional classes. The first class consists of three genes (raxA, raxB and raxC) that encode components of a bacterial type I secretion system (TOSS) (da Silva, F. G. et al., Mol Plant Microbe Interact 17:593 (2004)). The second class is involved in sulfation, including raxST, which encodes a protein with similarity to mammalian tyrosine sulfotransferases (da Silva, F. G. et al., Mol Plant Microbe Interact 17:593 (2004)). Xoo strains carrying mutations in any of these rax genes no longer activate XA21-mediated immunity. None of the identified genes encode an obvious activator of immunity.

To identify Ax21, we fractionated the supernatant of Xoo strain PXO99 cultures on a C18 reverse phase-high performance liquid chromatography (RP-HPLC) column (FIG. 1A), and carried out bio-assays of seven HPLC peptide-enriched fractions (FIG. 1B) using our previously established methods (Lee, S.-W. et al., Proc Natl Acad Sci USA 103:18395 (2006)).

An active fraction that was able to trigger XA21-mediated immunity (FIG. 1) was subjected to liquid chromatography-mass spectrometry (LC-MS/MS) (Sun, W. et al., Plant Cell 18:64 (2006)). Fifteen peptides from the LC-MS/MS spectra matched eight Xoo proteins (Sun, W. et al., Plant Cell 18:64 (2006)); including two peptides that corresponded to the N-terminal and C-terminal regions of a 194 aa protein encoded by PXO_{—}03968 (boxes in FIG. 1C and FIG. 5).

To identify which gene encodes Ax21, we generated Xoo strains carrying a mutation in each of the individual genes. Whereas a PXO_{—}03968 knockout strain caused long lesion and grew to high levels on XA21 leaves (FIG. 2), none of the other strains did (FIG. 6). These data indicate that the PXO_{—}03968 gene encodes Ax21. We further showed that Ax21 secretion requires raxA and raxC (FIG. 7) (Sun, W. et al., Plant Cell 18:64 (2006)).

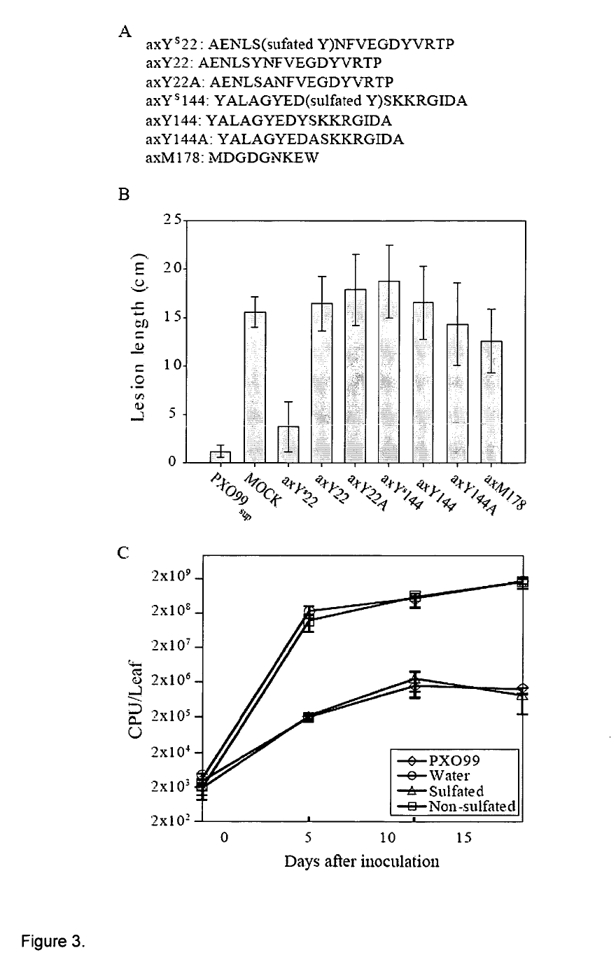

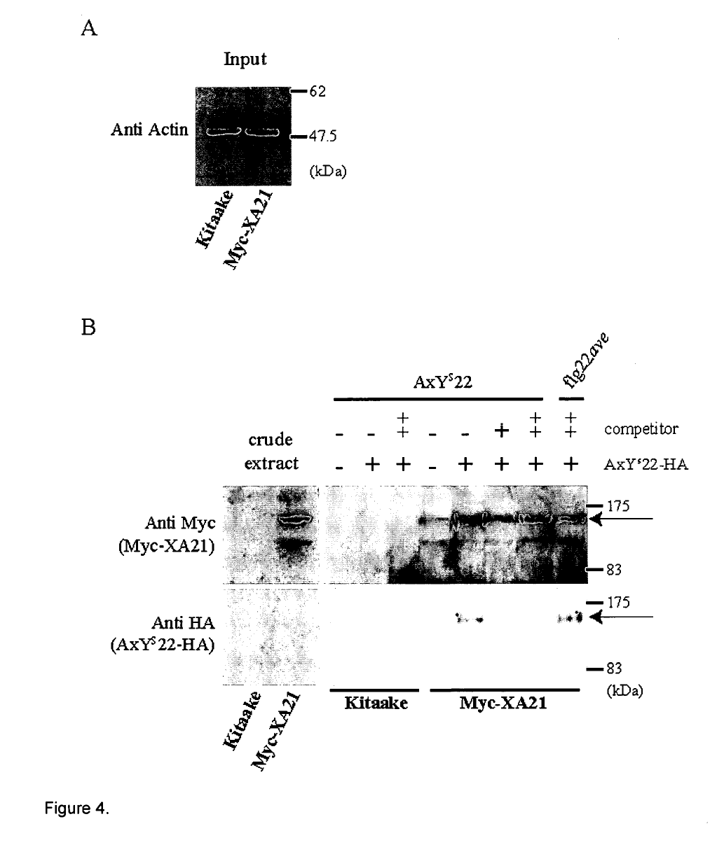

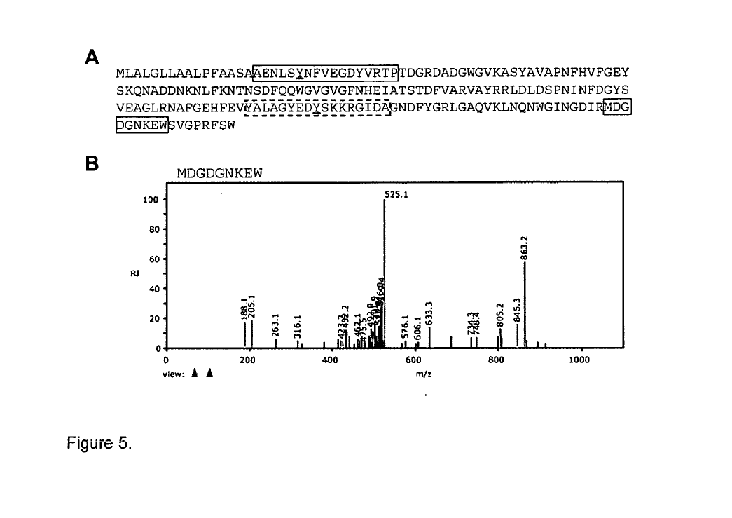

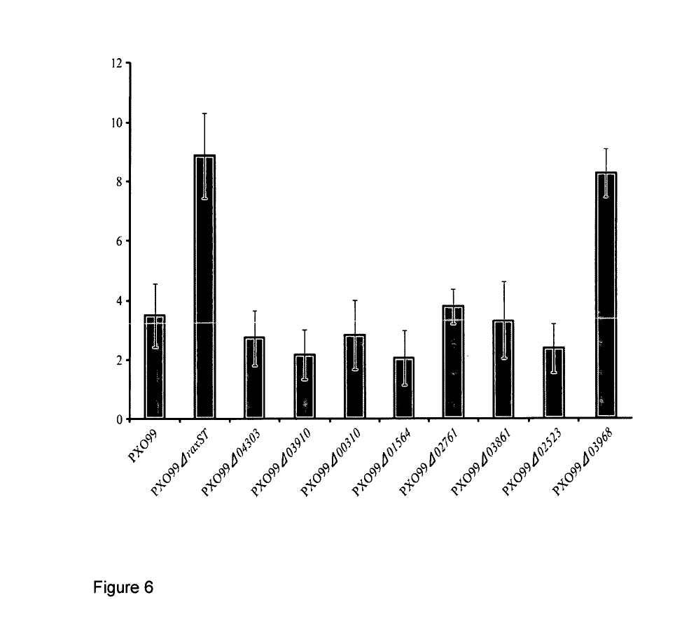

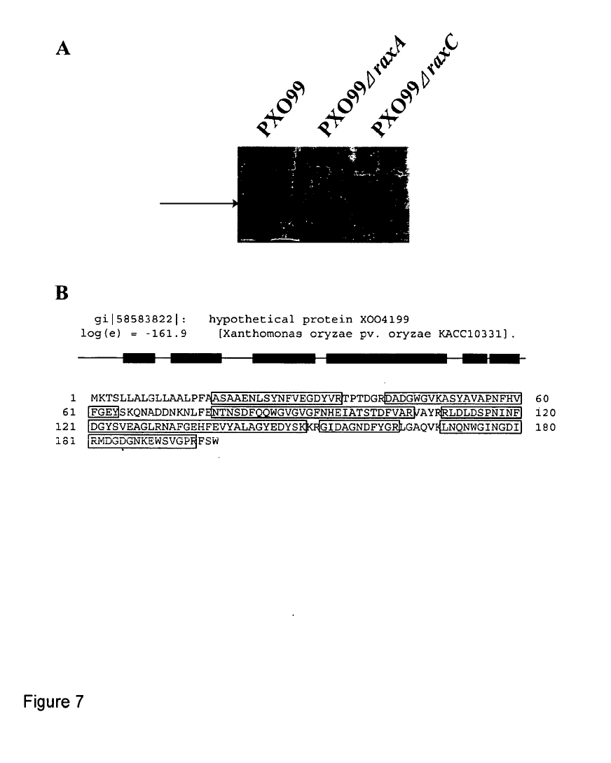

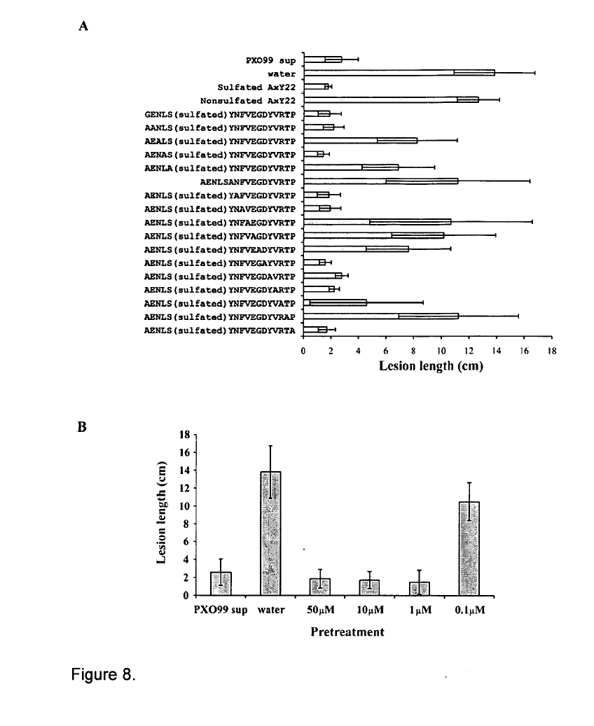

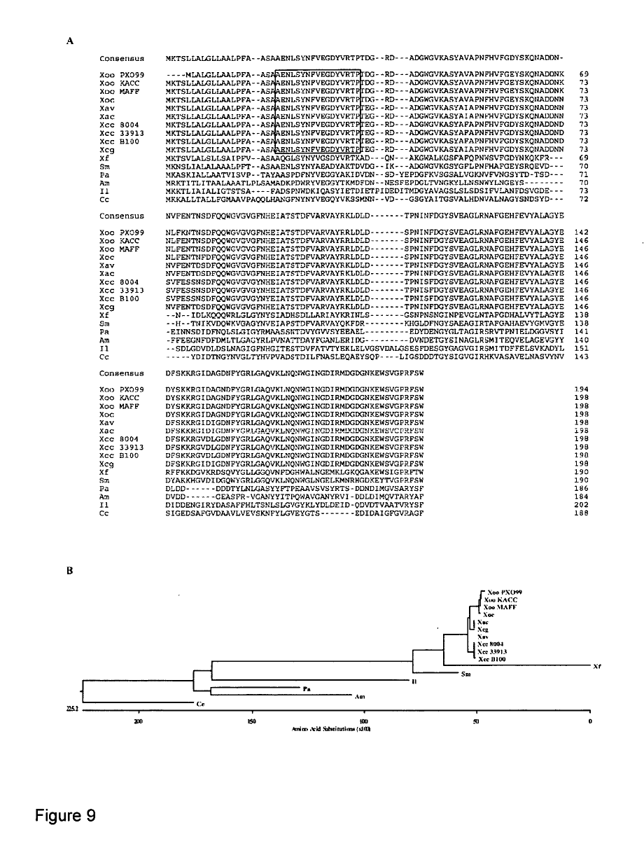

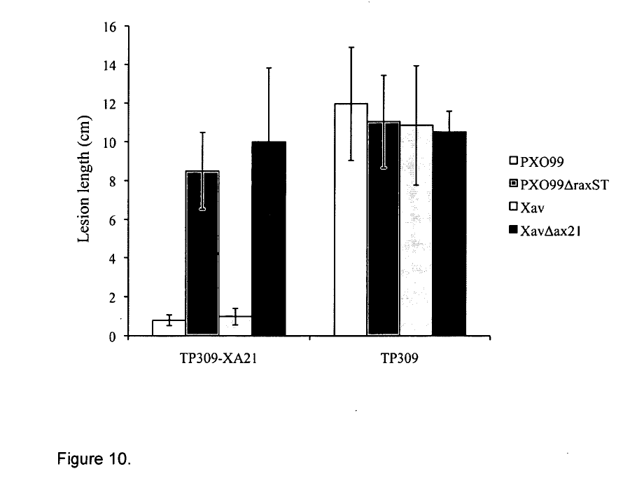

To test the importance of the putative tyrosine sulfation sites on Ax21 (See supporting material on Science Online), we synthesized seven peptides, two carrying sulfated tyrosines in the target residues (Y22 and Y144), two carrying non-sulfated tyrosines, two carrying alanines in place of the tyrosines, and one corresponding to the C-terminal region of Ax21 (FIG. 3A) (See supporting material on Science Online). XA21 rice leaves were pretreated with each peptide (100 μmol/ml in water). The 17 aa peptide carrying Y22 sulfation (axY^{S}22) activated XA21-mediated immunity (FIG. 3B). To further quantify this response, we characterized the activity of the axY^{S}22 synthetic peptide using growth curve analysis. Pretreatment of XA21 rice leaves with the axY^{S}22 peptide triggered resistance to PXO99ΔraxST as reflected in a 1000-fold reduction in PXO099ΔraxST population growth. The non-sulfated peptide (axY22) was unable to trigger Xa21-mediated immunity (FIG. 3C)

Bioassays with 17 AxY^{S}22 peptide variants carrying alanine substitutions identified eight amino acids critical for XA21-mediated immunity (FIG. 8A) (Sun, W. et al., Plant Cell 18:64 (2006)). A concentration of 1 μM is sufficient for PAMP activity (FIG. 8B) (Sun, W. et al., Plant Cell 18:64 (2006)).

In co-immunoprecipitation experiments with HA-tagged AxY^{S}22 and extracts from leaves carrying a Myc-tagged XA21 protein (Sun, W. et al., Plant Cell 18:64 (2006)), we observed labeling of a band migrating at 140 kDa on a SDS-PAGE gel with both anti-Myc and anti-HA antibodies (FIG. 4). The presence of 5- to 10-fold excess, untagged AxY^{S}22 peptide suppressed the labeling of this band, whereas flg22ₐᵥₑ from the rice pathogen Acidovorax avenae had no effect on AxY^{S}22/XA21 binding (FIG. 4). These experiments demonstrate that XA21 is required for AxY^{S}22 binding and recognition.

Sequence analysis indicates that Ax21 is highly conserved in Xoo strains (KACC 10331 and MAFF 311018, both 98% identity), X. campestris pv. campestris (90%), X. axonopodis pv. glycinea (92%), X. axonopodis pv. vesicatoria 85-10 (92%), and X. oryzae pv. oryzicola (98%) (FIG. 8). The 17 aa AxY^{S}22 sequence is 100% conserved in these strains. Xylella fastidiosa and the opportunistic human pathogen, Stenotrophomonas maltophilia also carry putative Ax21 orthologs (48% and 61%, respectively) and show 77% and 65%, respectively, to the AxY^{S}22 sequence (FIG. 9).

Because Xav carries predicted orthologs for Ax21, raxST, raxA and raxB, and because we have previously shown that these genes are required for Ax21 activity (da Silva, F. G. et al., Mol Plant Microbe Interact 17:593 (2004)), we hypothesized that Xav would express Ax21 activity. Indeed, we found that pretreatment of XA21 rice leaves with supernatants from Xav wild-type, but not Xav strains carrying a deletion of ax21 (Sun, W. et al., Plant Cell 18:64 (2006)), can activate XA21-mediated immunity (FIG. 10). These results indicate that the ax21 ortholog in Xav possesses the predicted biological activity.

One of the key aspects of the definition of PAMPs is that they “are conserved within a class of microbes” (Medzhitov, R., Nat Rev Immunol 1:135 (2001)). Due to the explosion of studies on PRRs and PAMPs in both plant and animal systems, it has now become clear that PAMPs can be conserved quite widely across genera (e.g. flagellin) or more narrowly within a genus (e.g., Pepl3) (Brunner, F. et al., EMBO J21:6681 (2002)) and, further, that sequence variation and post-translational modifications can modulate PRR-dependent pathogen recognition (Sun, W. et al., Plant Cell 18:64 (2006); Takeuchi, K. et al., J. Bacteriol. 185:6658 (2003)).

In the XA21/Ax21 system, the AxY^{S}22 peptide sequence is invariant in all sequenced Xanthomonas species. Sulfation provides specificity to the system, just as flagellin or lipopolysaccharide recognition in some hosts is modulated by glycosylation or acylation, respectively (Takeuchi, K. et al., J. Bacteriol. 185:6658 (2003); Doz, E. et al., J Biol Chem 282:26014 (2007)). Thus, Ax21 is a PAMP that satisfies the genetic definition of an avirulence factor because the presence or absence of sulfation on the conserved 17 aa epitope is decisive for its ability to trigger XA21-mediated immunity. Similarly, Xa21 is a disease resistance gene because it is the single polymorphic determinant in rice that confers resistance to strains of bacteria expressing sulfated Ax21, and it is also a PRR because it is required for recognition of a particular modified peptide epitope that is conserved across a microbial genus.

Thus, our data provide another example of bacterial-host interactions that can be attributed to the presence of genes encoding proteins (e.g., sulfotransferases, glycosylases, and acetylases) that modify conserved peptide epitopes (Takeuchi, K. et al., J. Bacteriol. 185:6658 (2003); Doz, E. et al., J Biol Chem 282:26014 (2007); Silipo, A. et al., Chembiochem 9:896 (2008); Carneiro, L. A. et al., Microbes Infect 6:609 (2004); Wolfert, M. A. et al., Infect Immun 75:706 (2007)). Such examples indicate that successful pathogens of plants and animals have evolved methods of altering the PAMP to avoid detection by the host PRR. Conversely, the presence or absence of a particular PRR can have a dramatic effect on the resistance of the host to infection. Just as plants deficient in XA21 or FLS2 exhibit reduced resistance to phytopathogens, mice deficient for TLR4 or TLR2 are altered in their response to Mycobacterium tuberculosis infection (Doz, E. et al., J Biol Chem 282:26014 (2007)). These studies have led to a convergence in our understanding of the molecular mechanisms governing the specificity of host-microbe interactions in plants and animals.

### Supplemental Materials

### 1. LC-MSMS Analysis of the Ax21-Active Fraction

LC-MSMS analysis of the Ax21-active fraction revealed the presence of peptides corresponding to eight proteins in addition to Ax21. These included a phospholipid-binding protein encoded by PXO_{—}04303 (locus tag No. in PXO99 genome database), a YceI-like protein by PXO_{—}03910, a putative lipoprotein by PXO_{—}00310, TolB by PXO_{—}01564, a peptidyl dipeptidase by PXO_{—}02761, a secreted xylanase by PXO_{—}03861, an outer membrane protein by PXO_{—}02523 and a hypothetical protein by PXO_{—}03968. Lesion length analysis of XA21 rice plants inoculated with Xoo strains deleted for each of these genes revealed that only PXO_{—}03968 conferred Ax21 activity (FIG. 7).

2. AX21 Secretion Requires a TOSS Encoded by raxA and raxC

To determine if Ax21 requires the RaxABC Type one system for secretion, we carried out sodium dodecyl sulfate polyacrylamide gel electrophoresis (SDS-PAGE) on peptides extracted from supernatants of PXO99, PXO99ΔraxA and PXO99ΔraxC strains. We identified a single band present in strain PXO99 but absent in PXO99ΔraxA and PXO99ΔraxC (FIG. 7A). MS analysis of this band revealed 6 peptides that matched AX21 with 68% coverage (FIG. 7B). These data indicate that AX21 secretion requires raxA and raxC, consistent with the requirement of these two genes for Ax21 activity. The program “Sulfinator” (F. Monigatti, E. Gasteiger, A. Bairoch, E. Jung, Bioinformatics 18, 769 (2002)) identified two candidate tyrosine sulfation targets (Y22 and Y144) on the Ax21 protein (underlined Ys in FIG. 2).

### 3. Xav Strain 8510 Carries Functional Ax21 Ortholog

To confirm that the Ax21 homolog of Xav is required for AX21 PAMP activity, we pretreated TP309-XA21 and TP309 rice leaves with supernatants from Xav and an Xav mutant strain carrying a deletion of Ax21 (XavAax2l) (FIG. 10). Supernatants from PXO99 and PXO99ΔraxST were used as a positive and negative control. Pretreated leaves were inoculated five hours later with PXO99ΔraxST. Ax21 activity was evaluated by measuring lesion lengths three weeks after inoculation (FIG. 10). These results indicate that the Ax21 ortholog in Xav is required for Ax21 activity and is specific to XA21 rice plants.

### 4. AxY^{S}22 but not AxY22 Triggers Immunity in XA21 Rice.

Our quantitative lesion length analysis indicated that pretreatment with the AxY^{S}22 peptide was sufficient to activate XA21 mediated immunity (FIG. 4B). To further quantify these results, we carried out bacterial growth curve analysis of rice XA21 leaves pretreated with the AxY^{S}22 and AxY22 peptides. Pretreatment with supernatant from PXO99 and water were used as a positive and negative control. Leaves cut at the tip from six-week-old TP309-XA21 rice plants (at the 5-leaf stage) were dipped into each solution containing 100 μM of AxY^{S}22 and AxY22, the supernatant from PXO99, and water for 5 hours, and then scissor-inoculated with the PXO99ΔraxST strain immediately below the first cut site as described above. Bacterial growth curve were established at 0, 5, 10, and 15 days after inoculation (FIG. 4C). These results indicate that AxY^{S}22 but not AxY22 triggers immunity in TP309-XA21 rice. Thus, a peptide variant lacking sulfation has no detectable activity.

### 5. Alanine-Scanning Mutagenesis of the AxY^{S}22 Peptide.

We generated 17 AxY^{S}22 peptide variants carrying alanine (glycine for the first residue) substitutions and tested their activity (FIG. 8A). TP309-XA21 rice plants were pretreated with solutions containing 100 μM of each of the AxY^{S}22 peptide variants. Following pretreatment, PXO99ΔraxST was inoculated using the scissor clipping method immediately below the first cut site. Ax21 activity was evaluated by measuring lesion lengths three weeks after PXO99ΔraxST strain inoculation (FIG. 8A). Peptides carrying substitutions in amino acids 19 (asparagine), 21 (serine), 22 (tyrosine), 25 (valine), 26 (glutamate), 27 (glycine), or 32 (threonine) failed to induce XA21-mediated immunity. Substitution of amino acid 31 (arginine) partially disrupted activity.

### 6. μM of the AxY^{S}22 Peptide is Sufficient for Biological Activity.

TP309-XA21 rice plants were pretreated with solutions containing 50, 10, 1, or 0.1 μM of the AxY^{S}22 peptide. Supernatants from PXO99 and water were used as positive and negative controls, respectively. Following pretreatment, PXO99ΔraxST was inoculated using the scissor-clipping method immediately below the first cut site. Ax21 activity was evaluated by measuring lesion lengths three weeks after PXO99ΔraxST strain inoculation (FIG. 8B). 1 μM of the AxY^{S}22 peptide is sufficient for the PAMP activity. In rice 0.5 μM of flg22 from Acidovorax avenae can induce OsFLS2-mediated response (R. Takai, A. Isogai, S. Takayama, F.-S. Che., Mol Plant Microbe Interact 21, 1635 (2008)). Similarly in Arabidopsis, 1 μM of elf26 or 1 μM of flg22 can induce resistance to subsequent infection by Pseudomonas syringae pv. tomato (Kunze et al., 2004). Whereas elf26 induces only partial resistance, Ax21 induces robust resistance, similar to the resistance induced by flg22.

### Materials and Methods

### Biological Materials and Growth Conditions.

Xanthomonas oryzae pv. oryzae (Xoo) Philippine race 6 strain PXO99Az (provided by Jan Leach and called PXO99 in this study) and X. axonopodis pv. vesicatoria (Xav) strain 85-10 were used in this report. Other Xoo and Escherichia coli strains and plasmids used in this study are listed in Table S1. Peptone sucrose media (PS) (K. Tsuchiya, T. M. Mew, S. Wakimoto, Phytopathology 72, 43 (1982)) containing 20 μg/mL of cephalexin (MP Biomedicals) and/or other antibiotics as appropriate were used for growing cultures of Xoo and Xav at 28° C. E. coli strains were cultured in Luria-Bertani (LB) medium at 37° C. For E. coli, kanamycin at 50 μg/mL, ampicillin at 50 μg/ml, and gentamycin at 25 μg/mL (15 μg/mL for Xoo and Xav) were used for selection of transformants. For inoculation experiments, the Oryza sativa ssp. japonica rice varieties Taipei 309 (TP309) (a rice line lacking XA21) and the 106-17-3-37 (TP309-XA21) transgenic line (W. Song et al., Science 270, 1804 (1995)) was used. A Kitaake transgenic line carrying Myc-XA21 (C. J. Park et al., submitted in Plant Cell, (2009)) and a Kitaake control were used for the XA21-Ax21 binding assay.

TABLE S1
Bacterial strains and plasmids used in this study

| | | |
|---|---|---|
| | | Source or |
| Strain or plasmid | Relevant characteristics | reference |

Escherichia coli

| | | |
|---|---|---|
| DH10B | F⁻ mcrAΔ (mrr-hsdRMS-mcrBC) | Gibco BRL |
| | Φ80lacZΔM15ΔlacX74 deoR | |
| | recA1 endA1 araΔ 139 | |
| | Δ (ara, leu) 7697ga1U galKλ⁻ | |
| | rpsL(Sm′) nupGλ⁻ tonA | |

Xanthomonas oryzae pv. oryzae

| | | |
|---|---|---|
| PXO99 | Philippine race 6 (PR6) strain, Cp′ | |
| PXO99ΔraxST | PXO99 raxST::Km, Ax21⁻, Km′ | |
| PXO99ΔraxA | PXO99 raxA::Km, Ax21⁻, Km′ | |
| PXO99ΔraxC | PXO99 raxA::Km, Ax21⁻, Km′ | |
| PXO99Δax21 | PXO99 03968::Km, Ax21⁻, Km′ | This study |

Xanthomonas axonopodis pv. vesicatoria

| | |
|---|---|
| 85-10 | wild type, Cp′ |
| XavΔax21 | Xav 0208::Km, Ax21⁻, Km′ |

Plasmids

| | | |
|---|---|---|
| pGEM ®-T | pGEM ®-5Zf(+) ori, Ap′ | Promega Corp., |
| | | Madison, WI, |
| | | U.S.A. |
| pGEM ®-TAx21 | pGEM-T carrying a 854-bp fragment | This study |
| | containing part of the PXO_03968 | |
| | ORF, disrupted by a Km′ cassett | |

Cp′: Cephalexin resistance,
Km′: Kanamycin resistance

Peptide extraction. Twenty liters of Xoo cells cultured in PS broth medium at 28° C. for 3 days were harvested with centrifugation (10,000 g for 10 min). After washing with 1% glucose three times, the Xoo cells were resuspended in 1 L of water medium containing 100 mM glucose and 10 mM sodium sulfate, incubated at 28° C. for 2 days. The cell-free supernatant was then separated by centrifugation (10,000 g for 10 min) followed by filtration using two layer of membrane filter (0.22 μm). The cell-free supernatant was concentrated by evaporation with butanol (less than 5%), resuspended with 100 mL of 50% ethanol containing 2% acetic acid, extracted with dichloromethane (1:1 ratio), eluted through a polyamide column, concentrated again (without butanol), resuspended in 50 mL of 50 mM NH₄HCO₃, applied to Sep-Pak® Vac 3cc C18 Cartridges, eluted 500 μl of with 80% ethanol and concentrated, and resuspended with acetonitrile (ACN) containing 0.1% (v/v) of trifluoroacetic acid (TFA).

Reverse phase-high pressure liquid chromatography (RP-HPLC) fractionation. Fifty μl of the peptide-enriched samples were fractionated on a reverse-phase HPLC column (Vydac TP C18, 1×250 mm, 300 Angstrom) with an ACN gradient containing 0.1% (v/v) TFA (10% at 15 min, 10 to 90% in 80 min, 90% at 5 min, then 90 to 10% at 5 min; 110 min total running time). The flow rate of the column was 50 μl per minutes. Eluates were fractionated every 10 min after sample injection using a fraction collector. Each fraction from 10 time HPLC elutions was evaporated and re-suspended with 10 mL of autoclaved deionized water and then use in the Ax21 activity assay.

Sodium dodecyl sulfate polyacrylamide gel electrophoresis (SDS-PAGE). Secreted peptides and small proteins were extracted for SDS-PAGE analysis from ten liters of each Xoo strain (PXO99, PXO99ΔraxA, and PXO99ΔraxC) culture using the method described above. The extracts were separated on a 12% SDS-acrylamide gel after quantification (100 μg for PXO99 and 500 μg for PXO99ΔraxA and PXO99ΔraxC) and stained with Silver stain plus (Bio-Rad). The band in the PXO99 extract, which was lacking in the PXO99ΔraxA and PXO99ΔraxC strain extracts (FIG. 7A), was subjected to liquid chromatography-mass spectrometry (LC-MS/MS) after trypsin digestion. Seven of peptides, all corresponding to the Ax21 protein were revealed by this analysis (FIG. 7B).

Mass spectrometery analysis. Peptides were analyzed by Liquid Chromatography (LC)-Mass spectrometry analysis on a linear trap quadrupole (LTQ) with Michrom Paradigm LC and CTC Pal autosampler (CTC Analytics). Peptides were separated with a 45 min gradient using a Michrom 200 μm×150 mm Magic C18AQ reversed phase column at 2 μl/min. Peptides were directly loaded onto a Agilent ZORBAX 300SB C18, reversed phase trap cartridge, which, after loading, was switched in-line with a Michrom Magic C18 AQ 200 μm×150 mm C18 column connected to a Thermo-Finnigan LTQ (Linear Trap Quadruple) iontrap mass spectrometer through a Michrom Advance Plug and Play nano-spray source. Peptides were separated using a gradient of 2-40% (A=0.1% Formic Acid, B=100% Acetonitrile) in 35 minutes, 40-80% in 1 minute, hold 1 minute, then 80-2% in 8 minutes (45 minute total run time). MS and MS/MS spectra were acquired using a top 10 method, where the top 10 ions in the MS scan were subjected to automated low energy collision induced dissociation. Peptide sequences of MS/MS data were obtained by de novo sequencing using X!Tandem, PEAKS Online 2.0 (http://www.bioinfor.com:8080/peaksonline/search.jsp), and Xcalibur. The Xoo genome database were searched with these peptide sequences (S. L. Salzberg et al., BMC Genomics 9, 204 (2008)).

Inoculation and Ax21 activity bioassays. Following surface sterilization, TP309 and TP309-XA21 (line 106-17-3-37) seeds were germinated in distilled water at 28° C. for four days, then planted into soil and grown for 6 weeks in a green house. Six-week old plants were transferred to a growth chamber at least two days prior to inoculation. The chamber conditions were as follows: 16/8 h day/night, 28/26° C., 80/90% relative humidity. Xoo cells were prepared by culturing on PS agar plates containing either cephalexin (for the PXO99 wild-type strain) or kanamycin (for the PXO99ΔraxST and PXO99ΔAx21 mutant strains) for three days at 28° C.

For lesion length analyses, rice leaves were inoculated with the scissors clipping method (W. Y. Song et al., Science 270, 1804 (1995)), using cells suspended in distilled water at a density of 108 CFU (colony forming unit) /mL. Lesion lengths were measured 12 days after inoculation. The results represent the averages of measurements taken from more than ten inoculated leaves per strain and each experiment was repeated 5 times.

For growth curve analyses, inoculated rice leaves were harvested at five time points (0, 3, 6, 9, and 12 days after inoculation or 0, 5, 10, and 15 days after PXO99ΔraxST inoculation following pretreatment, immediately sliced into small pieces, incubated in 5 ml sterile water including 15 μg/mL of cephalexin with shaking for 1 h, and then filtered through two layers of gauze. The filtrates were then plated onto PS agar plates with serial dilution. Colonies on the plates were counted after three days of incubation at 28° C.

For Ax21 activity bioassays, TP309-XA21 leaves were cut 3 cm below the tip and then soaked in a supernatant from a Xoo strain culture, a HPLC fraction or a suspension of synthetic peptides. After 5 hours of pretreatment, the leaves were inoculated with a PXO99ΔraxST strain suspension (108 CFU/mL) using scissors to cut directly below the pretreatment site. Lesion development was monitored for two to three weeks. The results in FIG. 4C represent the averages of measurements taken from 5 or 6 inoculated leaves. Each experiment was repeated twice.

Xoo and Xav gene replacements. Knock-out mutants were generated using our established marker exchange mutagenesis method (S.-W. Lee, P. C. Ronald, in Methods in Molecular Biology/Molecular Medicine (Humana Press, Inc., Totowa, N.J., 2006), pp. 11-17), a kanamycin-resistance (KmR) cassette (from pUK-4K), and the suicide vector pGEM®-T easy. DNA fragments for homologous recombination of the Xoo genes were synthesized using the PCR method with Taq polymerase in a Programmable Thermal Controller (MJ Research Inc.). Primer sequences for the eight genes are listed in Table S2. The kanamycin resistance cassette was inserted into appropriate restriction enzyme cleavage sites in the genes of interest. The constructs carrying the interrupted genes were then introduced into competent Xoo PXO99 or Xav 85-10 cells. After electroporation, the cells were incubated for 3 h at 28° C., and then spread on PS agar plates containing 50 μg/ml of kanamycin. Colonies that grew on those plates were re-plated onto PS agar plates containing 50 μg/ml of kanamycin as well as plates containing 50 μg/ml of kanamycin/ampicillin in order to select for double crossing-over events. Colonies which grew on the kanamycin-only plates, but not on kanamycin/ampicillin, were collected and confirmed to be insertional mutants using PCR with the same primers used for cloning.

TABLE S2
Primer sets for PCR cloning for candidates

| Gene | Primer sequence for 5′ (SEQ ID NO:) | | Primer sequence for 3′ (SEQ ID NO:) | |
|---|---|---|---|---|
| PXO_04303 | 5′-TGAAAACGGTTCATCGCAAC-3′ | (55) | 5′-CAGACAAGTCCTGTTGAACCA-3′ | (56) |
| PXO_03910 | 5′-AAGACCACCCACAAGCTGTT-3′ | (57) | 5′-TTACTTGGCTGAGGCATCCTT-3′ | (58) |
| PXO_00310 | 5′-CATCACCACACGCATTTCAA 3′ | (59) | 5′-TGATACGGCATTACTTGGCCT-3′ | (60) |
| PX0_01564 | 5′-GAAGAGACGGCGTACAGCA-3′ | (61) | 5′-TTACCAGGGCTCCGACACT-3′ | (62) |
| PX0_02761 | 5′-ATGTCGCGTACCGTCGTT-3′ | (63) | 5′-GTGGCGGCGTAGAACACG-3′ | (64) |
| PXO_03861 | 5′-ATGTTGAAACTCCGTTACCCG-3′ | (65) | 5′-TGGCAACGTAGCTGCGTA-3′ | (66) |
| PX0_02525 | 5′-TGGAAGCGATCTCGGTGAAT-3′ | (67) | 5′-AACCGGCCAGGACTAACTTA-3′ | (68) |
| PXO_03968 | 5′-GAGAGAGTCGCCTTGCAGTT-3′ | (69) | 5′-AGCGCTAGAGCGTCACATTT-3′ | (70) |
| Xav 0208 | 5′-GAGAGAGTCGCCTTGCAGTT-3′ | (71) | 5′-AGCGCTAGAGCGTCACATTT-3′ | (72) |

### Ax21/XA21 Binding Assays

Binding of the C terminal HA tagged AxY^{S}22 (AxY^{S}22-HA) to N-terminal tagged Myc XA21 (Myc-XA21) complex was carried out using cross-linked analysis according to methods described previously (C. Zipfel et al., Cell 125, 749 (2006); Z. Bauer, L. Gomez-Gomez, T. Boller, G. Felix, J Biol Chem 276, 45669 (2001); D. Chinchilla, Z. Bauer, M. Regenass, T. Boller, G. Felix, Plant Cell 18, 465 (2006)). Fully expanded rice leaves were harvested from five-week old Myc-XA21 transgenic and Kitaake control plants. Total proteins were extracted from five g of leaf tissue in 10 ml of ice-cold extraction buffer [0.15 M NaCl, 0.01 M Na-phosphate pH 7.2, 2 mM EDTA, 0.1% Triton X-100, 10 mM β-mercaptoethanol, 20 mM NaF, 1 mM PMSF, 1% Protease cocktail (Sigma), 2 μg/ml leupeptin, 2 μg/ml antipain, and 2 μg/ml aprotinin] After filtration through Miracloth (Calbiochem) followed by centrifugation twice at 13,000 g for 20 min at 4° C., the supernatants were incubated in a total volume of 500 μl with AxY^{S}22-HA for 30 min either alone or with different concentrations of competitor [AxY^{S}22 (no HA tag) or flg22 from Acidovorax avenae (QRLSSGLRINSAKDDAAGLAIS; SEQ ID NO:54) (R. Takai, A. Isogai, S. Takayama, F. S. Che, Mol Plant Microbe Interact 21, 1635 (2008) peptides]. After binding of AxY^{S}22-HA with Myc-XA21, cross-linking was initiated by addition of 25 μl of 100 mM ethylene glycol bis (succinimidylsuccinate) (Pierce) in DMSO directly to the incubation mixture. After further incubation for 1 h on ice, the reaction was quenched by the addition of 10 μl lo f 2 M Tris-HCl, pH 7.5. Immunoprecipitation of Myc-XA21 was performed as described previously (C. J. Park et al., PLoS Biology 6, e231 (2008)). Forty microliters of agarose conjugated anti-Myc antibody (Santa Cruz) was added into the binding mixture of Myc-XA21 and AxY^{S}22-HA and incubated at 4° C. for 1 h. The beads were then washed four times in 1 ml of extraction buffer without proteinase inhibitors. The proteins were eluted with 4× Laemmli loading buffer. Duplicate protein gel blots were analyzed with anti-Myc and anti-HA antibodies, respectively. Myc-XA21 and a proteolytic cleavage product of Myc-XA21 displayed bands at about 140 and 110 kDa, respectively, as reported previously (C. J. Park et al., PLoS Biology 6, e231 (2008); Y. S. Wang et al., Plant Cell 18, 3635 (2006); W. H. Xu et al., Plant J45, 740 (2006)).

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. An isolated or purified polypeptide comprising A(E/Q)(N/G)LSY*N(F/Y)(V/A)(E/G)(G/A/S)DY(V/A)(R/K)T(P/D/K) (SEQ ID NO:2), wherein Y* represents a sulfated tyrosine and wherein the amino acids in parentheses are options at the designated position.

2. The polypeptide of claim 1, consisting of A(E/Q)(N/G)LSY*N(F/Y)(V/A)(E/G)(G/A/S)DY(V/A)(R/K)T(P/D/K) (SEQ ID NO:2), wherein Y* represents a sulfated tyrosine.

3. The polypeptide of claim 1, comprising AENLSY*NFVEGDYVRTP (SEQ ID NO:1), wherein Y* represents a sulfated tyrosine.

4. The polypeptide of claim 1, consisting of AENLSY*NFVEGDYVRTP (SEQ ID NO:1), wherein Y* represents a sulfated tyrosine.

5. The polypeptide of claim 1, wherein the polypeptide, when contacted to a rice plant expressing XA21, enhances disease resistance in the plant compared to a control plant not contacted with the polypeptide.

6.-18. (canceled)

19. A plant contacted with an exogenous application of the polypeptide of claim 1.

20. The plant of claim 19, wherein the plant is a seed.

21. The plant of claim 19, wherein the plant is a rice plant.

22. The plant of claim 19, wherein the plant expresses XA21.

23.-30. (canceled)

31. An isolated host cell comprising a heterologous expression cassette, the expression cassette comprising a promoter operably linked to a polynucleotide, the polynucleotide encoding a polypeptide comprising A(E/Q)(N/G)LSYN(F/Y)(V/A)(E/G)(G/A/S)DY(V/A)(R/K)T(P/D/K) (SEQ ID NO:2).

32. The host cell of claim 31, wherein the polypeptide consists of A(E/Q)(N/G)LSYN(F/Y)(V/A)(E/G)(G/A/S)DY(V/A)(R/K)T(P/D/K) (SEQ ID NO:2).

33. The host cell of claim 31, wherein the polypeptide comprises AENLSYNFVEGDYVRTP (SEQ ID NO:1).

34. The host cell of claim 31, wherein the polypeptide consists of AENLSYNFVEGDYVRTP (SEQ ID NO:1).

35. The host cell of any of claims 31 31 claim 31,wherein the host cell expresses the polypeptide and the polypeptide comprises a sulfated tyrosine.

36. The host cell of claim 31, selected from the group consisting of a plant cell, a fungal cell, a bacterial cell, a yeast cell, a insect cell and a mammalian cell.


SEQUENCE LISTING (72 sequences)

SEQ ID NO: 1 - protein, 17 aa, Artificial Sequence
  synthetic activator of Xa21-mediated immunity (Ax21, avirulenceXa21, avrXa21) N-terminal region, axS-Y22 peptide, sulfated peptide sufficient to induce XA21-mediated disease resistance
AENLSXNFVEGDYVRTP

SEQ ID NO: 2 - protein, 17 aa, Artificial Sequence
  synthetic activator of Xa21-mediated immunity (Ax21, avirulenceXa21, avrXa21) N-terminal region, axS-Y22 peptide, sulfated peptide sufficient to induce XA21-mediated disease resistance
AZXLSXNXXXXDYXXTX

SEQ ID NO: 3 - protein, 17 aa, Artificial Sequence
  synthetic activator of Xa21-mediated immunity (Ax21, avirulenceXa21, avrXa21) N-terminal region from heterologous expression cassette
AZXLSYNXXXXDYXXTX

SEQ ID NO: 4 - protein, 17 aa, Artificial Sequence
  synthetic activator of Xa21-mediated immunity (Ax21, avirulenceXa21, avrXa21) N-terminal region from heterologous expression cassette, axY22 with non-sulfated Tyr
AENLSYNFVEGDYVRTP

SEQ ID NO: 5 - protein, 194 aa, Xanthomonas oryzae
  Xanthomonas oryzae pv. oryzae (Xoo) Philippine race 6 strain PXO99Az activator of Xa21-mediated immunity (Ax21, avirulenceXa21, avrXa21)
MLALGLLAALPFAASAAENLSXNFVEGDYVRTPTDGRDADGWGVKASYAVAPNFHVFGEYSKQNADDNKNLFKNTNSDFQQWGVGVGFNHEIATSTDFVARVAYRRLDLDSPNINFDGYSVEAGLRNAFGEHFEVYALAGYEDXSKKRGIDAGNDFYGRLGAQVKLNQNWGINGDIRMDGDGNKEWSVGPRFSW

SEQ ID NO: 6 - protein, 17 aa, Artificial Sequence
  synthetic activator of Xa21-mediated immunity (Ax21, avirulenceXa21, avrXa21) N-terminal region with Ala in place of Tyr, axY22A peptide
AENLSANFVEGDYVRTP

SEQ ID NO: 7 - protein, 17 aa, Artificial Sequence
  synthetic activator of Xa21-mediated immunity (Ax21, avirulenceXa21, avrXa21) central region, axS-Y144 peptide
YALAGYEDXSKKRGIDA

SEQ ID NO: 8 - protein, 17 aa, Artificial Sequence
  synthetic activator of Xa21-mediated immunity (Ax21, avirulenceXa21, avrXa21) central region, axY144 peptide with non-sulfated Tyr
YALAGYEDYSKKRGIDA

SEQ ID NO: 9 - protein, 17 aa, Artificial Sequence
  synthetic activator of Xa21-mediated immunity (Ax21, avirulenceXa21, avrXa21) central region with Ala in place of Tyr, axY144A peptide
YALAGYEDASKKRGIDA

SEQ ID NO: 10 - protein, 9 aa, Artificial Sequence
  synthetic activator of Xa21-mediated immunity (Ax21, avirulenceXa21, avrXa21) C-terminal region, axM178 peptide
MDGDGNKEW

SEQ ID NO: 11 - protein, 198 aa, Xanthomonas oryzae
  Xanthomonas oryzae pv. oryzae (Xoo) Philippine race 6 strain PXO99Az activator of Xa21-mediated immunity (Ax21, avirulenceXa21, avrXa21)
MKTSLLALGLLAALPFAASAAENLSYNFVEGDYVRTPTDGRDADGWGVKASYAVAPNFHVFGEYSKQNADDNKNLFENTNSDFQQWGVGVGFNHEIATSTDFVARVAYRRLDLDSPNINFDGYSVEAGLRNAFGEHFEVYALAGYEDYSKKRGIDAGNDFYGRLGAQVKLNQNWGINGDIRMDGDGNKEWSVGPRFSW

SEQ ID NO: 12 - protein, 17 aa, Artificial Sequence
  synthetic alanine scanning mutagenesis AxS-Y22 peptide variant
GENLSXNFVEGDYVRTP

SEQ ID NO: 13 - protein, 17 aa, Artificial Sequence
  synthetic alanine scanning mutagenesis AxS-Y22 peptide variant
AANLSXNFVEGDYVRTP

SEQ ID NO: 14 - protein, 17 aa, Artificial Sequence
  synthetic alanine scanning mutagenesis AxS-Y22 peptide variant
AEALSXNFVEGDYVRTP

SEQ ID NO: 15 - protein, 17 aa, Artificial Sequence
  synthetic alanine scanning mutagenesis AxS-Y22 peptide variant
AENASXNFVEGDYVRTP

SEQ ID NO: 16 - protein, 17 aa, Artificial Sequence
  synthetic alanine scanning mutagenesis AxS-Y22 peptide variant
AENLAXNFVEGDYVRTP

SEQ ID NO: 17 - protein, 17 aa, Artificial Sequence
  synthetic alanine scanning mutagenesis AxS-Y22 peptide variant
AENLSANFVEGDYVRTP

SEQ ID NO: 18 - protein, 17 aa, Artificial Sequence
  synthetic alanine scanning mutagenesis AxS-Y22 peptide variant
AENLSXAFVEGDYVRTP

SEQ ID NO: 19 - protein, 17 aa, Artificial Sequence
  synthetic alanine scanning mutagenesis AxS-Y22 peptide variant
AENLSXNAVEGDYVRTP

SEQ ID NO: 20 - protein, 17 aa, Artificial Sequence
  synthetic alanine scanning mutagenesis AxS-Y22 peptide variant
AENLSXNFAEGDYVRTP

SEQ ID NO: 21 - protein, 17 aa, Artificial Sequence
  synthetic activator of Xa21-mediated immunity (Ax21, avirulenceXa21, avrXa21) N-terminal region, axS-Y22 peptide
AENLSXNFVAGDYVRTP

SEQ ID NO: 22 - protein, 17 aa, Artificial Sequence
  synthetic alanine scanning mutagenesis AxS-Y22 peptide variant
AENLSXNFVEADYVRTP

SEQ ID NO: 23 - protein, 17 aa, Artificial Sequence
  synthetic alanine scanning mutagenesis AxS-Y22 peptide variant
AENLSXNFVEGAYVRTP

SEQ ID NO: 24 - protein, 17 aa, Artificial Sequence
  synthetic alanine scanning mutagenesis AxS-Y22 peptide variant
AENLSXNFVEGDAVRTP

SEQ ID NO: 25 - protein, 17 aa, Artificial Sequence
  synthetic alanine scanning mutagenesis AxS-Y22 peptide variant
AENLSXNFVEGDYARTP

SEQ ID NO: 26 - protein, 17 aa, Artificial Sequence
  synthetic alanine scanning mutagenesis AxS-Y22 peptide variant
AENLSXNFVEGDYVATP

SEQ ID NO: 27 - protein, 17 aa, Artificial Sequence
  synthetic activator of Xa21-mediated immunity (Ax21, avirulenceXa21, avrXa21) N-terminal region, axS-Y22 peptide
AENLSXNFVEGDYVRAP

SEQ ID NO: 28 - protein, 17 aa, Artificial Sequence
  synthetic alanine scanning mutagenesis AxS-Y22 peptide variant
AENLSXNFVEGDYVRTA

SEQ ID NO: 29 - protein, 194 aa, Xanthomonas oryzae
  Xanthomonas oryzae pv. oryzae (Xoo) Philippine race 6 strain PXO99Az activator of Xa21-mediated immunity (Ax21, avirulenceXa21, avrXa21)
MLALGLLAALPFAASAAENLSYNFVEGDYVRTPTDGRDADGWGVKASYAVAPNFHVFGEYSKQNADDNKNLFKNTNSDFQQWGVGVGFNHEIATSTDFVARVAYRRLDLDSPNINFDGYSVEAGLRNAFGEHFEVYALAGYEDYSKKRGIDAGNDFYGRLGAQVKLNQNWGINGDIRMDGDGNKEWSVGPRFSW

SEQ ID NO: 30 - protein, 198 aa, Xanthomonas oryzae; identical to SEQ ID NO: 11
  Xanthomonas oryzae pv. oryzae (Xoo) strain KACC activator of Xa21-mediated immunity, Ax21 ortholog

SEQ ID NO: 31 - protein, 198 aa, Xanthomonas oryzae; identical to SEQ ID NO: 11
  Xanthomonas oryzae pv. oryzae (Xoo) strain MAFF activator of Xa21-mediated immunity, Ax21 ortholog

SEQ ID NO: 32 - protein, 198 aa, Xanthomonas oryzae
  Xanthomonas oryzae pv. oryzicola (Xoc) AAQN00000000, Xoryp_01570 activator of Xa21-mediated immunity, Ax21 ortholog
MKTSLLALGLLAALPFAASAAENLSYNFVEGDYVRTPTDGRDADGWGVKASYAVAPNFHVFGEYSKQNADDNNNLFENTNFDFQQWGVGVGFNHEIATSTDFVARVAYRRLDLDSPNINFDGYSVEAGLRNAFGEHFEVYALAGYEDYSKKRGIDAGNDFYGRLGAQVKLNQNWGINGDIRMDGDGNKEWSVGPRFSW

SEQ ID NO: 33 - protein, 198 aa, Xanthomonas axonopodis
  Xanthomonas axonopodis pv. vesicatoria (Xav) AM039952, XCV0208 activator of Xa21-mediated immunity, Ax21 ortholog
MKTSLLALGLLAALPFAASAAENLSYNFVEGDYVRTPTEGRDADGWGVKASYAIAPNFHVFGDYSKQNADDNNNVFENTDSDFQQWGVGVGFNHEIATSTDFVARVAYRKLDLDTPNINFDGYSVEAGLRNAFGEHFEVYALAGYEDFSKKRGIDIGDNFYGRLGAQVKLNQNWGINGDIRMDGDGNKEWSVGPRFSW

SEQ ID NO: 34 - protein, 198 aa, Xanthomonas axonopodis; identical to SEQ ID NO: 33
  Xanthomonas axonopodis pv. citri (Xac) AE008923, XAC0223 activator of Xa21-mediated immunity, Ax21 ortholog

SEQ ID NO: 35 - protein, 198 aa, Xanthomonas campestris
  Xanthomonas campestris pv. campestris 8004 (Xcc 8004) CP000050, ACC0205 activator of Xa21-mediated immunity, Ax21 ortholog
MKTSLLALGLLAALPFAASAAENLSYNFVEGDYVRTPTEGRDADGWGVKASYAFAPNFHVFGDYSKQNADDNDSVFESSNSDFQQWGVGVGYNHEIATSTDFVARVAYRKLDLDTPNISFDGYSVEAGLRNAFGEHFEVYALAGYEDFSKKRGVDLGDNFYGRLGAQVKLNQNWGINGDIRMDGDGNKEWSVGPRFSW

SEQ ID NO: 36 - protein, 198 aa, Xanthomonas campestris; identical to SEQ ID NO: 35
  Xanthomonas campestris pv. campestris 33913 (Xcc 33913) AE008922 activator of Xa21-mediated immunity, Ax21 ortholog

SEQ ID NO: 37 - protein, 198 aa, Xanthomonas campestris
  Xanthomonas campestris pv. campestris B100 (Xcc B100) AM920689, XCCB100_0226 activator of Xa21-mediated immunity, Ax21 ortholog
MKTSLLALGLLAALPFAASAAENLSYNFVEGDYVRTPTEGRDADGWGVKASYAFAPNFHVFGDYSKQNADDNDSVFESSNSDFQQWGVGVGYNYEIATSTDFVARVAYRKLDLDTPNISFDGYSVEAGLRNAFGEHFEVYALAGYEDFSKKRGVDLGDNFYGRLGAQVKLNQNWGINGDIRMDGDGNKEWSVGPRFSW

SEQ ID NO: 38 - protein, 198 aa, Xanthomonas axonopodis; identical to SEQ ID NO: 33
  Xanthomonas axonopodis pv. glycines (Xag) AAS91338 activator of Xa21-mediated immunity, Ax21 ortholog

SEQ ID NO: 39 - protein, 190 aa, Xylella fastidiosa
  Xylella fastidiosa (Xf) AAAL00000000, XfasaDRAFT_1077 activator of Xa21-mediated immunity, Ax21 ortholog
MKTSVLALSLLSAIPFVASAAQGLSYNYVGSDYVRTKADQNAKGWALKGSFAFQPNWSVFGDYNKQKFRNIDLKQQQWRLGLGYNYSIADHSDLLARIAYKRINLSGSNPNSNGINPEVGLNTAFGDHALVYTLAGYERFFKKDGVKRDSQVYGLLGGQVNFDGHWALNGEMKLGKQGAKEWSIGPRFTW

SEQ ID NO: 40 - protein, 190 aa, Stenotrophomonas maltophilia
  Stenotrophomonas maltophilia (Sm) AM743169, Smlt0387 activator of Xa21-mediated immunity, Ax21 ortholog
MKNSLIALALAAALPFTASAAENLSYNYAEADYAKTDVDGIKADGWGVKGSYGFLPNFHAFGEYSRQEVDHTNIKVDQWKVGAGYNVEIAPSTDFVARVAYQKFDRKHGLDFNGYSAEAGIRTAFGAHAEVYGMVGYEDYAKKHGVDIDGQWYGRLGGQVKLNQNWGLNGELKMNRHGDKEYTVGPRFSW

SEQ ID NO: 41 - protein, 186 aa, Pseudoalteromonas atlantica
  Pseudoalteromonas atlantica (Pa) ABG38916, Patl_0386 activator of Xa21-mediated immunity, Ax21 ortholog
MKASKIALLAATVISVPTAYAASPDFNYVEGGYAKIDVDNSDYEPDGFKVSGSALVGKNVFVNGSYTDTSDEINNSDIDFNQLSLGIGYRMAASSNTDVYGVVSYEEAELEDYDENGYGLTAGIRSRVTPNIELDGGVSYIDLDDDDDTYLNLGASYYFTPEAAVSVSYRTSDDNDIMGVSARYSF

SEQ ID NO: 42 - protein, 184 aa, Alteromonas macleodi
  Alteromonas macleodi (Am) ACG68266, MADE_03976 activator of Xa21-mediated immunity, Ax21 ortholog
MRKTITLITAALAAATLPLSAMADKPDWRYVEGGYTKMDFDNNESFEPDGLTVNGKYLLNSNWYLNGEYSFFEEGNFDFDMLTLGAGYRLPVNATTDAYFGANLERIDGDVNDETGYSINAGLRSMITEQVELAGEVGYYDVDDGEASFRVGANYYITPQWAVGANYRVIDDLDIMQVTARYAF

SEQ ID NO: 43 - protein, 202 aa, Idiomarina loihiensis
  Idiomarina loihiensis (Il) L2TR, AAV82257, IL1417 activator of Xa21-mediated immunity, Ax21 ortholog
MKKTLIAIALIGTSTSAFADSPNWDKIQASYIETDIETPIDEDITMDGYAVAGSLSLSDSIFVLANFDSVGDESDLGDVDLDSLNAGIGFNHGITESTDVFATVTYEKLELVGSVDALGSESFDESGYGAGVGIRSMITDFFELSVKADYLDIDDENGIRYDASAFFHLTSNLSLGVGYKLYDLDEIDQDVDTVAATVRYSF

SEQ ID NO: 44 - protein, 188 aa, Chlorobium chlorochromatii
  Chlorobium chlorochromatii (Cc) CaD3, ABB27819, Cag_0546 activator of Xa21-mediated immunity, Ax21 ortholog
MKKALLTALLFGMAAVPAQQLHANGFNYNYVEGQYVKSSMNNVDGSGYAITGSVALHDNVALNAGYSNDSYDYDIDTNGYNVGLTYHVPVADSTDILFNASLEQAEYSQPLIGSDDDTGYSIGVGIRHKVASAVELNASVYNVSIGEDSAFGVDAAVLVEVSKNFYLGVEYGTSEDIDAIGFGVRAGF

SEQ ID NO: 45 - protein, 197 aa, Artificial Sequence
  synthetic activator of Xa21-mediated immunity, Ax21 ortholog consensus sequence
MKTSLLALGLLAALPFAASAAENLSYNFVEGDYVRTPTDGRDADGWGVKASYAVAPNFHVFGDYSKQNADDNNVFENTNSDFQQWGVGVGFNHEIATSTDFVARVAYRKLDLDTPNINFDGYSVEAGLRNAFGEHFEVYALAGYEDFSKKRGIDAGDNFYGRLGAQVKLNQNWGINGDIRMDGDGNKEWSVGPRFSW

SEQ ID NO: 46 - protein, 16 aa, Artificial Sequence
  synthetic activator of Xa21-mediated immunity (Ax21, avirulenceXa21, avrXa21) active fragment
ZXLSXNXXXXDYXXTX

SEQ ID NO: 47 - protein, 15 aa, Artificial Sequence
  synthetic activator of Xa21-mediated immunity (Ax21, avirulenceXa21, avrXa21) active fragment
XLSXNXXXXDYXXTX

SEQ ID NO: 48 - protein, 16 aa, Artificial Sequence
  synthetic activator of Xa21-mediated immunity (Ax21, avirulenceXa21, avrXa21) active fragment
AZXLSXNXXXXDYXXT

SEQ ID NO: 49 - protein, 15 aa, Artificial Sequence
  synthetic activator of Xa21-mediated immunity (Ax21, avirulenceXa21, avrXa21) active fragment
AZXLSXNXXXXDYXX

SEQ ID NO: 50 - protein, 16 aa, Artificial Sequence
  synthetic activator of Xa21-mediated immunity (Ax21, avirulenceXa21, avrXa21) active fragment
ENLSXNFVEGDYVRTP

SEQ ID NO: 51 - protein, 15 aa, Artificial Sequence
  synthetic activator of Xa21-mediated immunity (Ax21, avirulenceXa21, avrXa21) active fragment
NLSXNFVEGDYVRTP

SEQ ID NO: 52 - protein, 16 aa, Artificial Sequence
  synthetic activator of Xa21-mediated immunity (Ax21, avirulenceXa21, avrXa21) active fragment
AENLSXNFVEGDYVRT

SEQ ID NO: 53 - protein, 15 aa, Artificial Sequence
  synthetic activator of Xa21-mediated immunity (Ax21, avirulenceXa21, avrXa21) active fragment
AENLSXNFVEGDYVR

SEQ ID NO: 54 - protein, 22 aa, Acidovorax avenae
  Acidovorax avenae flg22
QRLSSGLRINSAKDDAAGLAIS

SEQ ID NO: 55 - DNA, 20 bp, Artificial Sequence
  synthetic PCR cloning primer sequence for 5′ PXO_04303
tgaaaacggttcatcgcaac

SEQ ID NO: 56 - DNA, 21 bp, Artificial Sequence
  synthetic PCR cloning primer sequence for 3′ PXO_04303
cagacaagtcctgttgaacca

SEQ ID NO: 57 - DNA, 20 bp, Artificial Sequence
  synthetic PCR cloning primer sequence for 5′ PXO_03910
aagaccacccacaagctgtt

SEQ ID NO: 58 - DNA, 21 bp, Artificial Sequence
  synthetic PCR cloning primer sequence for 3′ PXO_03910
ttacttggctgaggcatcctt

SEQ ID NO: 59 - DNA, 20 bp, Artificial Sequence
  synthetic PCR cloning primer sequence for 5′ PXO_00310
catcaccacacgcatttcaa

SEQ ID NO: 60 - DNA, 21 bp, Artificial Sequence
  synthetic PCR cloning primer sequence for 3′ PXO_00310
tgatacggcattacttggcct

SEQ ID NO: 61 - DNA, 19 bp, Artificial Sequence
  synthetic PCR cloning primer sequence for 5′ PXO_01564
gaagagacggcgtacagca

SEQ ID NO: 62 - DNA, 19 bp, Artificial Sequence
  synthetic PCR cloning primer sequence for 3′ PXO_01564
ttaccagggctccgacact

SEQ ID NO: 63 - DNA, 18 bp, Artificial Sequence
  synthetic PCR cloning primer sequence for 5′ PXO_02761
atgtcgcgtaccgtcgtt

SEQ ID NO: 64 - DNA, 18 bp, Artificial Sequence
  synthetic PCR cloning primer sequence for 3′ PXO_02761
gtggcggcgtagaacacg

SEQ ID NO: 65 - DNA, 21 bp, Artificial Sequence
  synthetic PCR cloning primer sequence for 5′ PXO_03861
atgttgaaactccgttacccg

SEQ ID NO: 66 - DNA, 18 bp, Artificial Sequence
  synthetic PCR cloning primer sequence for 3′ PXO_03861
tggcaacgtagctgcgta

SEQ ID NO: 67 - DNA, 20 bp, Artificial Sequence
  synthetic PCR cloning primer sequence for 5′ PXO_02525
tggaagcgatctcggtgaat

SEQ ID NO: 68 - DNA, 20 bp, Artificial Sequence
  synthetic PCR cloning primer sequence for 3′ PXO_02525
aaccggccaggactaactta

SEQ ID NO: 69 - DNA, 20 bp, Artificial Sequence
  synthetic PCR cloning primer sequence for 5′ PXO_03968
gagagagtcgccttgcagtt

SEQ ID NO: 70 - DNA, 20 bp, Artificial Sequence
  synthetic PCR cloning primer sequence for 3′ PXO_03968
agcgctagagcgtcacattt

SEQ ID NO: 71 - DNA, 20 bp, Artificial Sequence
  synthetic PCR cloning primer sequence for 5′ Xav 0208
gagagagtcgccttgcagtt

SEQ ID NO: 72 - DNA, 20 bp, Artificial Sequence
  synthetic PCR cloning primer sequence for 3′ Xav 0208
agcgctagagcgtcacattt